(19) United States
(12) Patent Application Publication
(10) Pub. No.: US 2024/0294570 A1
(43) Pub. Date: Sep. 5, 2024

(54) ENZYMATICALLY FORMING INTRANUCLEAR PEPTIDE ASSEMBLIES FOR SELECTIVELY KILLING INDUCED PLURIPOTENT STEM CELLS

(71) Applicants: BRANDEIS UNIVERSITY, Waltham, MA (US); HEBECELL CORP., Natick, MA (US)

(72) Inventors: Shuang LIU, Waltham, MA (US); Shijiang LU, Shrewsbury, MA (US); Bing XU, Newton, MA (US)

(21) Appl. No.: 18/573,504

(22) PCT Filed: Jun. 22, 2022
(86) PCT No.: PCT/US2022/034482

§ 371 Date: Dec. 22, 2023

Related U.S. Application Data
(60) Provisional application No. 63/213,484, filed on Jun. 22, 2021.

(51) Int. Cl. C07K 7/02; A61K 9/51; A61K 38/00

(52) U.S. Cl. CPC C07K 7/02; A61K 9/51; A61K 38/00

(57) ABSTRACT

An isolated peptide having the structure: where —NH-Q-C(O)— is an α-helical amino acid sequence that includes at least 4 and up to 30 amino acid residues, Z₁ is a moiety including an aromatic group or a fluorophore, Z₂ includes a phosphorylated amino acid residue, or the dephosphorylated amino acid residue. These peptides, in dephosphorylated form, are capable of self-assembly in the form of a nanoribbon, nanofiber, or a combination thereof. Such peptides are capable of causing cell death of cells that overexpress alkaline phosphatases, which includes cancer cells and induced pluripotent stem cells (iPSCs), and therefore the peptides of the invention can be used ex vivo to selectively cause cell death of cancer cells or iPSCs, or in vivo to cause cancer cell death.

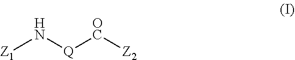

This application claims the priority benefit of U.S. Provisional Patent Application Ser. No. 63/213,484 filed Jun. 22, 2021, which is hereby incorporated by reference in its entirety.

This invention was made with government support under grant number CA142746 awarded by the National Institutes of Health and grant number DMR-2011846 awarded by the National Science Foundation. The government has certain rights in the invention.

### FIELD OF THE INVENTION

The invention relates to isolated peptides and compositions, and their use in methods of causing cell death, specifically causing selective cell death of induced pluripotent stem cells even when present in mixed populations of cells.

### BACKGROUND

Having unprecedented potential to generate a variety of cell types for cell therapy, pluripotent stem cells (PSCs), such as human embryonic stem cells (ESC) and human induced pluripotent stem cells (iPSCs), promise to revolutionize personalized medicine (Takahashi et al., “Induction of Pluripotent Stem Cells from Adult Human Fibroblasts by Defined Factors,” Cell 131(5):861-872 (2007)). Increased research efforts have focused on the use of PSCs in clinical applications, such as PSC-derived dopamine (DA) neurons for treating Parkinson's disease (Kriks et al., “Dopamine Neurons Derived from Human ES Cells Efficiently Engraft in Animal Models of Parkinson's Disease,” Nature 480 (7378):547-51 (2011)), differentiating PSC into cardiomyocytes (Zhang et al., “Functional Cardiomyocytes Derived from Human Induced Pluripotent Stem Cells,” Circ. Res. 104(4):e30-e41 (2009) or epicardium cells (Witty et al., “Generation of the Epicardial Lineage from Human Pluripotent Stem Cells,” Nat. Biotechnol. 32(10): 1026-35 (2014)) for treating heart disease, the generation of insulin-producing pancreatic beta cells from PSCs for treating diabetes (Pagliuca et al., “Generation of Functional Human Pancreatic Beta Cells in vitro,” Cell 159(2):428-39 (2014)), photoreceptor progenitors derived from PSCs for treating blindness (Barnea-Cramer et al., “Function of Human Pluripotent Stem Cell-derived Photoreceptor Progenitors in Blind Mice,” Sci. Rep. 6:29784 (2016)), and PSC-derived retinal pigment epithelium (RPE) for treating age-related macular degeneration (AMD) (Sharma et al., “Clinical-grade Stem Cell-derived Retinal Pigment Epithelium Patch Rescues Retinal Degeneration in Rodents and Pigs,” Sci. Transl. Med. 11(475):eaat5580 (2019)). Despite the rapid progresses of iPSC technology, considerable challenges remain to be met before safe clinical applications of PSCs (Yamanaka, “Pluripotent Stem Cell-Based Cell Therapy—Promise and Challenges,” Cell Stem Cell 27(4):523-531 (2020); Harding et al., “Preclinical Studies for Induced Pluripotent Stem Cell-based Therapeutics,” J. Biol. Chem 289(8):4585-4593 (2014)). Because a key feature of iPSCs is their potential for infinite proliferation, one major safety concern of iPSCs is their tumorigenicity (Knoepfler, “Deconstructing Stem Cell Tumorigenicity: A Roadmap to Safe Regenerative Medicine,” Stem Cells 27(5): 1050-1056 (2009)). For example, undifferentiated iPSCs exhibit comparable tumor producing potential with that of Hela cells in a rat model (Kanemura et al., “Tumorigenicity Studies of Induced Pluripotent Stem Cell (iPSC)-derived Retinal Pigment Epithelium (RPE) for the Treatment of Age-Related Macular Degeneration,” PLOS ONE 9(1):e85336 (2014)). Several studies even have shown a small number of residual iPSCs may produce teratomas in animals (Kawamata et al., “Design of a Tumorigenicity Test for Induced Pluripotent Stem Cell (iPSC)-Derived Cell Products,” J. Clin. Med. 4(1): 159-71 (2015); Liu et al., “The Tumourigenicity of iPS Cells and Their Differentiated Derivates,” J. Cell Mol. Med. 17(6): 782-91 (2013)). Moreover, inefficient differentiation protocols, variability of differentiation efficiency, or heterogeneity of iPSC clones all can lead to residual or large numbers of undifferentiated iPSCs after the differentiation procedure (Hu et al., “Neural Differentiation of Human Induced Pluripotent Stem Cells Follows Developmental Principles But With Variable Potency,” Proc. Natl. Acad. Sci. USA 107(9):4335-40 (2010)). Thus, it is necessary to eliminate the undifferentiated iPSCs without harming differentiated cells in a cell mixture prior to cell transplantation.

Considerable efforts have been spent on developing approaches to eliminate residual undifferentiated iPSCs (Schuldiner et al., “Selective Ablation of Human Embryonic Stem Cells Expressing a ‘Suicide’ Gene,” Stem Cells 21(3):257-265 (2003); Choo et al., “Selection Against Undifferentiated Human Embryonic Stem Cells By a Cytotoxic Antibody Recognizing Podocalyxin-like protein-1,” Stem Cells 26(6): 1454-1463 (2008); Tateno et al., “Elimination of Tumorigenic Human Pluripotent Stem Cells By a Recombinant Lectin-Toxin Fusion Protein,” Stem Cell Rep. 4(5):811-820 (2015); Tateno, “Development of Lectin-drug Conjugates for Elimination of Undifferentiated Cells and Cancer Therapy,” Trends Glycosci. Glycotechnol. 31(183):e121-E127 (2019); Fong et al., “Separation of SSEA-4 and TRA-1-60 Labelled Undifferentiated Human Embryonic Stem Cells from a Heterogeneous Cell Population Using Magnetic-Activated Cell Sorting (MACS) and Fluorescence-Activated Cell Sorting (FACS),” Stem Cell Rev. 5(1): 72-80 (2009); Menendez et al., “Increased Dosage of Tumor Suppressors Limits the Tumorigenicity of iPS Cells Without Affecting Their Pluripotency,” Aging Cell 11(1):41-50 (2012); Miki et al., “Efficient Detection and Purification of Cell Populations Using Synthetic MicroRNA Switches,” Cell Stem Cell 16(6): 699-711 (2015); Matsumoto et al., “Plasma-activated Medium Selectively Eliminates Undifferentiated Human Induced Pluripotent Stem Cells,” Regen. Ther. 5:55-63 (2016); Okada et al., “Selective Elimination of Undifferentiated Human Pluripotent Stem Cells Using Pluripotent State-specific Immunogenic Antigen Glypican-3,” Biochem. Biophys. Res. 511(3):711-717 (2019); Shiraki et al., “Methionine Metabolism Regulates Maintenance and Differentiation of Human Pluripotent Stem Cells,” Cell Metab. 19(5): 780-794 (2014); Nagashima et al., “Selective Elimination of Human Induced Pluripotent Stem Cells Using Medium with High Concentration of L-Alanine,” Sci. Rep. 8(1): 12427 (2018); Blum et al., “The Anti-apoptotic Gene Survivin Contributes to Teratoma Formation By Human Embryonic Stem Cells,” Nat. Biotechnol. 27(3):281-287 (2009); Ben-David et al., “Immunologic and Chemical Targeting of the Tight-junction Protein Claudin-6 Eliminates Tumorigenic Human Pluripotent Stem Cells,” Nat. Commun. 4:1992 (2013); Kuo et al., “Selective Elimination of Human Pluripotent Stem Cells by a Marine Natural Product Derivative,” J. Am. Chem. Soc. 136(28):9798-9801 (2014); Mao et al., “A Synthetic Hybrid Molecule for the Selective Removal of Human Pluripotent Stem Cells from Cell Mixtures,” Angew. Chem. Int. Ed. 56(7): 1765-1770 (2017); Mao et al., “Chemical Decontamination of iPS Cell-derived Neural Cell Mixtures,” Chem. Commun. 54(11): 1355-1358 (2018); Go et al., “Structure-activity Relationship Analysis of YM155 for Inducing Selective Cell Death of Human Pluripotent Stem Cells,” Front. Chem. 7:298 (2019); Kim et al., “Ethanol Extract of Magnoliae Cortex (EEMC) Limits Teratoma Formation of Pluripotent Stem Cells by Selective Elimination of Undifferentiated Cells Through the p53-dependent Mitochondrial Apoptotic Pathway,” Phytomedicine 69:153198 (2020); Kondo, “Selective Eradication of Pluripotent Stem Cells by Inhibiting DHODH Activity,” Stem Cells 39(1):33-42 (2021); Hermann et al., “Possible Applications of New Stem Cell Sources in Neurology,” Nervenarzt 84(8): 943-948 (2013); Rampoldi et al., “Targeted Elimination of Tumorigenic Human Pluripotent Stem Cells Using Suicide-Inducing Virus-like Particles,” ACS Chem. Biol. 13(8):2329-2338 (2018); Hong et al., “Suppression of Induced Pluripotent Stem Cell Generation By the p53-p21 Pathway,” Nature 460(7259): 1132-1135 (2009)) for the safe clinical applications of iPSC-based cell therapy, but current strategies still have many drawbacks. Therefore, there still exists a need to develop an innovative strategy that is rapid (≤2 hours), effective, and general for eliminating undifferentiated iPSCs in cell mixture.

A prominent difference between iPSCs and differentiated cells is that iPSCs overexpress (or upregulate) alkaline phosphatase (ALP) (Stefkova et al., “Alkaline Phosphatase in Stem Cells,” Stem Cells Int 2015:628368 (2015)), but the differentiated cells do not. Thus, it is possible to selectively kill iPSCs by using enzyme-instructed self-assembly (EISA) (Yang et al., “Enzymatic Formation of Supramolecular Hydrogels,” Adv. Mater. 16(16): 1440-1444 (2004); He et al., “Enzymatic Noncovalent Synthesis for Mitochondrial Genetic Engineering of Cancer Cells,” Cell Rep. Phys. Sci. 1(12): 100270 (2020); Feng et al., “Enzyme-Instructed Peptide Assemblies Selectively Inhibit Bone Tumors,” Chem 5(9):2442-2449 (2019); Wang et al., “Intercellular Instructed-Assembly Mimics Protein Dynamics To Induce Cell Spheroids,” J. Am. Chem. Soc. 141(18):7271-7274 (2019); Yan et al., “Activatable NIR Fluorescence/MRI Bimodal Probes for in Vivo Imaging by Enzyme-Mediated Fluorogenic Reaction and Self-Assembly,” J. Am. Chem. Soc. 141(26): 10331-10341 (2019); Yang et al., “Desuccinylation-Triggered Peptide Self-Assembly: Live Cell Imaging of SIRT5 Activity and Mitochondrial Activity Modulation,” J. Am. Chem. Soc. 142(42): 18150-18159 (2020); Cheng et al., “Autocatalytic Morphology Transformation Platform for Targeted Drug Accumulation,” J. Am. Chem. Soc. 141(10):4406-4411 (2019); Wang et al., “A Photoacoustic Probe for the Imaging of Tumor Apoptosis by Caspase-Mediated Macrocyclization and Self-Assembly,” Angew. Chem. Int. Ed. 58(15):4886-4890 (2019); Tanaka et al., “Cancer Cell Death Induced by the Intracellular Self-Assembly of an Enzyme-Responsive Supramolecular Gelator,” J. Am. Chem. Soc. 137(2):770-775 (2015); Chen et al., “Exploring the Condensation Reaction between Aromatic Nitriles and Amino Thiols To Optimize In Situ Nanoparticle Formation for the Imaging of Proteases and Glycosidases in Cells,” Angew. Chem. Int. Ed. 59(8):3272-3279 (2020); Shi et al., “De novo Design of Selective Membrane-Active Peptides by Enzymatic Control of Their Conformational Bias on the Cell Surface,” Angew. Chem. Int. Ed. 58(39): 13706-13710 (2019); Wang et al., “Tumour Sensitization Via the Extended Intratumoural Release of a STING Agonist and Camptothecin from a Self-assembled Hydrogel,” Nat. Biomed. Eng. 4(11): 1090-1101 (2020)), a molecular process that integrates enzyme reactions and self-assembly and is known to selectively kill cells based on overexpression of enzymes (Tanaka et al., “Cancer Cell Death Induced by the Intracellular Self-Assembly of an Enzyme-Responsive Supramolecular Gelator,” J. Am. Chem. Soc. 137(2): 770-775 (2015); Yang et al., “Intracellular Enzymatic Formation of Nanofibers Results in Hydrogelation and Regulated Cell Death,” Advanced Materials 19(20):3152-3156 (2007); Kuang et al., “Pericellular Hydrogel/Nanonets Inhibit Cancer Cells,” Angew. Chem. Int. Ed. 53(31): 8104-8107 (2014); Pires et al., “Controlling Cancer Cell Fate Using Localized Biocatalytic Self-Assembly of an Aromatic Carbohydrate Amphiphile,” J. Am. Chem. Soc. 137(2):576-579 (2015)). In fact, Saito et al. recently reported selectively eliminating iPSCs by EISA of D-phosphotetrapeptides (Kuang et al., “Efficient, Selective Removal of Human Pluripotent Stem Cells via Ecto-Alkaline Phosphatase-Mediated Aggregation of Synthetic Peptides,” Cell Chem. Biol. 24(6):685-694.e4 (2017)). Taking the advantage that ALP acts as an ectophosphatase to form pericellular nanofibers of D-peptides, Kuang et al. (“Pericellular Hydrogel/Nanonets Inhibit Cancer Cells,” Angew. Chem. Int. Ed. 53(31):8104-8107 (2014))) have shown that ALP overexpressed on iPSCs dephosphorylates the D-phosphotetrapeptides (e.g., 1, see FIG. 2A) into hydrophobic peptides, which self-assemble on cell surface to induce cell death. They demonstrated that 2 hours of the D-phosphopeptide treatment eliminates iPSCs and prevents residual iPSC-induced teratoma formation in a mouse tumorigenicity assay (Kuang et al., “Efficient, Selective Removal of Human Pluripotent Stem Cells via Ecto-Alkaline Phosphatase-Mediated Aggregation of Synthetic Peptides,” Cell Chem. Biol. 24(6):685-694.e4 (2017)). Although that work confirms EISA as a feasible approach for selectively and rapidly eliminating residual iPSCs, there are still a number of drawbacks:
(i) The D-phosphotetrapeptide undergoes pericellular EISA, thus it is unable to utilize intracellular ALP (Thul et al., “A Subcellular Map of the Human Proteome,” Science 356(6340):eaal3321 (2017)); and
(ii) The D-peptides, being proteolytic resistant, self-assemble to form β-sheet, which is considered to be amyloidogenic (Haass et al., “Soluble Protein Oligomers in Neurodegeneration: Lessons from the Alzheimer's Amyloid Beta-Peptide,” Nat. Rev. Mol. Cell Biol. 8(2): 101-12 (2007); Nelson et al., “Structure of the Cross-beta Spine of Amyloid-like Fibrils,” Nature 435(7043):773-8 (2005)).
   It would be desirable, therefore, to develop agents and procedures that can overcome the above-identified deficiencies, and achieve selective and rapid elimination residual iPSCs.

The present invention is directed to overcoming these and other deficiencies in the art.

### SUMMARY OF THE DISCLOSURE

A first aspect of the invention relates to an isolated peptide including the structure below:

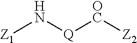

where
—NH-Q-C(O)— is an α-helical amino acid sequence including at least 4 and up to 30 amino acid residues,
Z₁ is a moiety including an aromatic group or a fluorophore, and
Z₂ including a phosphorylated amino acid residue, or the dephosphorylated amino acid residue.

A second aspect of the invention relates to supramolecular assembly of peptides according to the first aspect, where at least a portion of the peptides are dephosphorylated.

A third aspect of the invention relates to a pharmaceutical composition including one or more peptides according to the first aspect in an aqueous medium.

A fourth aspect of the invention relates to a method of causing cell death that includes the step of: contacting a cell that overexpresses a phosphatase with one or more peptides according to the first aspect, or a pharmaceutical composition according to the third aspect, which one or more peptides is phosphorylated, whereby the contacting step is effective to cause uptake of the one or more peptides and dephosphorylation of the phosphorylated amino acid residue(s) thereof by the phosphatase and thereby allow for intracellular self-assembly of the dephosphorylated one or more peptides.

A fifth aspect of the invention relates to a method for selectively causing cell death in a mixed population of cells that includes the steps of: providing a mixed population of cells including differentiated cells and one or more induced pluripotent stem cells; and contacting the mixed population of cells with one or more peptides according to the first aspect, or a pharmaceutical composition according to the third aspect, which one or more peptides is phosphorylated, whereby the contacting step is effective to cause uptake of the one or more peptides and dephosphorylation of the phosphorylated amino acid residue(s) thereof by a phosphatase overexpressed by the induced pluripotent stem cells, and thereby allow for intracellular self-assembly of the dephosphorylated one or more peptides in the induced pluripotent stem cells, but not differentiated cells, and selective induction of cell death in the induced pluripotent stem cells containing intracellular self-assemblies of the dephosphorylated one or more peptides.

As demonstrated in the accompanying Examples, an L-phosphopentapeptide (5), upon the dephosphorylation catalyzed by ALP, rapidly forms intranuclear peptide assemblies made of α-helix and aggregated strands that selectively kill iPSCs (see FIG. 1). Specifically, the phosphopentapeptide, containing four L-leucine residues and a C-terminal L-phosphotyrosine, self-assembles to form micelles or nanoparticles, which transform into peptide nanofibers or nanoribbons after enzymatic dephosphorylation removes the phosphate group from the phosphotyrosine. The concentrations of ALP dictate the morphology of the pentapeptide assemblies. While ALP of high expression level (800 U/L) catalyzes the dephosphorylation of the L-phosphopentapeptide to form nanoribbons, ALP of normal expression level (100 U/L) for the dephosphorylation results in nanofibers, depending on the time of incubation. Circular dichroism indicates that the L-pentapeptide adopts an α-helix and aggregated strand. Incubating the phosphopentapeptide with human iPSCs results in rapid killing of the iPSCs (=<2 h) due to the accumulation of the pentapeptide assemblies in the iPSC nuclei. The phosphopentapeptide is innocuous to normal cells (e.g., HEK293 and hematopoietic progenitor cell (HPC)). Inhibiting ALP abolishes the intranuclear assemblies. Mutating the L-phosphotyrosine from the C-terminal to the middle of the phosphopentapeptides or replacing L-leucine to D-leucine generates the phosphopentapeptides that undergo enzymatic self-assembly to form thin nanofibers. These two phosphopentapeptides are unable to result in intranuclear peptide assemblies for killing iPSCs. Treating the L-phosphopentapeptide with cell lysates of normal cells (e.g., HS-5) confirms the proteolysis of the L-pentapeptides. Compared to the previous approach described by Kuang et al. (“Efficient, Selective Removal of Human Pluripotent Stem Cells via Ecto-Alkaline Phosphatase-Mediated Aggregation of Synthetic Peptides,” Cell Chem. Biol. 24(6): 685-694.e4 (2017), which is hereby incorporated by reference in its entirety), there are several significant advancements:
(i) the L-phosphopeptides enter cells to undergo EISA to form intranuclear peptide assemblies;
(ii) L-peptide nanoribbons made of peptide α-helices minimize the risk of amyloidogenicity due to β-sheet; and
(iii) after killing the iPSCs, the L-peptides undergo proteolysis to form L-amino acids, thus minimizing side effects.
   This work, as the first case of intranuclear assemblies of peptides, illustrates the application of enzymatic noncovalent synthesis (He et al., “Enzymatic Noncovalent Synthesis,” Chem. Rev. 120(18):9994-10078 (2020), which is hereby incorporated by reference in its entirety) for selectively targeting nuclei of cells.

### BRIEF DESCRIPTION OF THE DRAWINGS

FIG. 1 is a schematic representation of EISA of L-phosphopentapeptide 5 to result in intranuclear assemblies of the de-phosphorylated pentapeptide 6.

FIG. 2A illustrates the chemical structure of a D-phosphotetrapeptide 1 and its de-phosphorylated D-tetrapeptide 2, reported in previous work by Kuang et al. (“Efficient, Selective Removal of Human Pluripotent Stem Cells via Ecto-Alkaline Phosphatase-Mediated Aggregation of Synthetic Peptides,” Cell Chem. Biol. 24(6):685-694.e4 (2017), which is hereby incorporated by reference in its entirety).

FIG. 2B illustrates the chemical structures of L-peptides and a D,L-peptide, whose phosphorylated forms are designated 3, 5, 7, 9 and de-phosphorylated forms are designated 4, 6, 8, 10. The structure of 3 and 4 is Xaa₁-Leu-Leu-Leu-Xaa₅ (SEQ ID NO: 1, where Xaa₁ is NBD-bAla and Xaa₅ is pTyr or Tyr). The structure of 5 and 6 is Xaa₁-Leu-Leu-Leu-Leu-Xaa₆ (SEQ ID NO: 2, where Xaa₁ is NBD-bAla and Xaa₆ is pTyr or Tyr). The structure of 7 and 9 is Xaa₁-Leu-Leu-Xaa₄-Leu-Leu (SEQ ID NO: 3, where Xaa₁ is NBD-bAla and Xaa₄ is pTyr or Tyr).

FIG. 3A is a panel of transmission electron microscope (TEM) images of 5, 7, 9 (400 μM, PBS) and the corresponding 6, 8, 10 formed by adding ALP (0.5 U/mL) for 24 h. FIG. 3B is a TEM image of 5 (400 μM, PBS) after dephosphorylation by ALP 0.8 U/mL for 2 h. The arrow shows the folding of nanoribbons, indicating that the self-assembly nanostructures are more like nanoribbons than nanotubes.

FIG. 4 is a panel of transmission electron microscope (TEM) images of 5 (400 μM, PBS) after dephosphorylation by ALP (0.1, 0.2, 0.4, 0.6, or 0.8 U/mL) for 1 or 2 h.

FIGS. 5A-5D are graphs illustrating the time-dependent dephosphorylation of 5, 7, and 9. FIGS. 5A-5B show dephosphorylation of 5 by ALP 0.5 U/mL and ALP 0.1 U/mL, respectively. FIG. 5C shows dephosphorylation of 7 by ALP 0.5 U/mL, and FIG. 5D shows dephosphorylation of 9 by ALP 0.5 U/mL.

FIGS. 6A-6B are graphs showing circular dichroism (CD) spectra of 5. FIG. 6A shows CD spectra of 5 (PBS) before and after dephosphorylation by 0.5 U/mL of ALP for 24 h (inset: magnified CD from 190-300 nm). FIG. 6B shows the time dependent CD spectra of 5 (100 μM, PBS) treated with 0.1 U/mL of ALP (inset: the molar ellipticity at 204 nm).

FIGS. 7A-7B are graphs showing CD spectra of 7 and 9 (PBS), respectively, before and after dephosphorylation by 0.5 U/mL ALP for 24 h.

FIG. 8A is a graph showing cell viability of iPSCs after incubating with 5, 7 or 9 for 2 h, ** p<0.01, *** p<0.001. FIG. 8B is a graph showing cell viability of iPS-derived HPCs, HS-5 cells, and HEK293 cells after incubating with 400 μM 5 for 2 h.

FIG. 9 is a panel of confocal laser scanning microscopy (CLSM) images of iPS cells after being treated by 5 (400 μM) for 2 h.

FIG. 10A is a final fluorescent image of time dependent CLSM of iPS cells treated by 5 (400 μM). FIG. 10B is a graph depicting the increase of mean fluorescent intensity of the areas A and B in FIG. 10A.

### DETAILED DESCRIPTION OF THE INVENTION

One aspect of the invention relates to a peptide capable of induced self-assembly having the structure

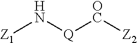

where
—NH-Q-C(O)— is an α-helical amino acid sequence, preferably including at least 4 and up to 30 amino acid residues, or optionally from 4 to 20 amino acid residues, from 4 to 15 amino acid residues, from 4 to 10 amino acid residues, or from 5 to 10 amino acid residues, or from 4 to 6 amino acid residues,
Z₁ is a moiety including an aromatic group or a fluorophore, and
Z₂ comprises a phosphorylated amino acid residue, or the dephosphorylated amino acid residue.

The isolated peptide, when exposed to a suitable phosphatase, is dephosphorylated at the Z₂ amino acid residue. This dephosphorylation of the Z₂ amino acid residue allows the peptides to self-assemble when present at a suitable concentration, thereby forming nanofibers and nanoribbons, as well as supramolecular assembles thereof. As used herein, the term “nanofibril” is defined as a fiber of material having any shape wherein at least one dimension, e.g. the diameter, width, thickness, and the like, is about 100 nm or less. Nanofibril diameters may be about 50 nm or less, about 40 nm or less, about 30 nm or less, about 20 nm or less, about 10 nm or less, about 5 nm or less, about 4 nm or less, about 3 nm or less, about 2 nm or less, or about 1 nm or less in diameter. Nanoribbons possess a uniquely sheet-like cross section such that the ribbons are wider than their thickness and much longer than their thickness. Although the peptides of the present invention, upon self-assembly, as described herein, form nanofibrils or nanoribbons, persons of skill in the art should appreciate that such peptides may also form microfibrils or microribbons that are larger than 100 nm thick.

As used herein, the term “amino acid” is intended to embrace all compounds, whether natural or synthetic, which include both an amino functionality and an acid functionality, including amino acid analogues and derivatives. In certain embodiments, the amino acids contemplated in the present invention are those naturally occurring amino acids found in proteins, or the naturally occurring anabolic or catabolic products of such amino acids, which contain amino and carboxyl groups. Amino acids, as used herein, may include both non-naturally and naturally occurring amino acids. The peptides preferably contain all L-amino acids.

The α-helical amino acid sequence, whose length is defined above, is preferably formed of multiple L-amino acid residues that promote the formation of the α-helix. It is well established that formation of a single turn of an α-helix requires 3.6 amino acid residues, in which case multiples thereof will dictate how many turns are present in the α-helical amino acid sequence.

The amino acid residues present in the α-helical amino acid sequence preferably include those that promote the formation of the α-helical amino acid sequence, although a minority of amino acid residues that are neutral or slightly interfere in α-helix formation may be tolerated. Particularly preferred are one or more amino acid residues independently selected from the group of alanine, alpha-aminobutyric acid, norvaline, valine, norleucine, isoleucine, and leucine.

In one embodiment, the α-helical amino acid sequence is the tetrapeptide

(SEQ ID NO: 4)
-AA₁-AA₂-AA₃-AA₄-

wherein each of AA₁, AA₂, AA₃, and AA₄ is independently selected from the group of alanine, alpha-aminobutyric acid, norvaline, valine, norleucine, isoleucine, and leucine.

In one embodiment, the α-helical amino acid sequence is the pentapeptide

(SEQ ID NO: 5)
-AA₁-AA₂-AA₃-AA₄-AA₅-

wherein each of AA₁, AA₂, AA₃, AA₄ and AA₅ is independently selected from the group of alanine, alpha-aminobutyric acid, norvaline, valine, norleucine, isoleucine, and leucine.

In one embodiment, the α-helical amino acid sequence is the hexapeptide

(SEQ ID NO: 6)
-AA₁-AA₂-AA₃-AA₄-AA₅-AA₆-

wherein each of AA₁, AA₂, AA₃, AA₄, AA₅, and AA₆ is independently selected from the group of alanine, alpha-aminobutyric acid, norvaline, valine, norleucine, isoleucine, and leucine.

In one embodiment, the α-helical amino acid sequence is the heptapeptide

(SEQ ID NO: 7)
-AA₁-AA₂-AA₃-AA₄-AA₅-AA₆-AA₇-

wherein each of AA₁, AA₂, AA₃, AA₄, AA₅, AA₆, and AA₇ is independently selected from the group of alanine, alpha-aminobutyric acid, norvaline, valine, norleucine, isoleucine, and leucine.

In one embodiment, the α-helical amino acid sequence is the octapeptide

(SEQ ID NO: 8)
-AA₁-AA₂-AA₃-AA₄-AA₅-AA₆-AA₇-AA₈-

wherein each of AA₁, AA₂, AA₃, AA₄, AA₅, AA₆, AA₇, and AA₈ is independently selected from the group of alanine, alpha-aminobutyric acid, norvaline, valine, norleucine, isoleucine, and leucine.

In one embodiment, the α-helical amino acid sequence is the nonapeptide

(SEQ ID NO: 9)
-AA₁-AA₂-AA₃-AA₄-AA₅-AA₆-AA₇-AA₈-AA₉-

wherein each of AA₁, AA₂, AA₃, AA₄, AA₅, AA₆, AA₇, AA₈, and AA₉ is independently selected from the group of alanine, alpha-aminobutyric acid, norvaline, valine, norleucine, isoleucine, and leucine.

In one embodiment, the α-helical amino acid sequence is the decapeptide

(SEQ ID NO: 10)
-AA₁-AA₂-AA₃-AA₄-AA₅-AA₆-AA₇-AA₈-AA₉-AA₁₀-

wherein each of AA₁, AA₂, AA₃, AA₄, AA₅, AA₆, AA₇, AA₈, AA₉, and AA₁₀ is independently selected from the group of alanine, alpha-aminobutyric acid, norvaline, valine, norleucine, isoleucine, and leucine.

Aromatic amino acids may optionally be present in the α-helical amino acid sequence or as an amino acid residue present within the Z₂ moiety. Such aromatic amino acids include phenylalanine, phenylalanine derivatives, napthylalanine, napthylalanine derivative, tyrosine, tyrosine derivatives, tryptophan, and tryptophan derivatives. In certain embodiments, aromatic residues of this type are present only in non-consecutive positions along the length of the α-helical amino acid sequence or along the length of the Z₂ moiety (or along the length of the peptide as a whole). For example, if multiple phenylalanine residues are present, there is no instance where Phe-Phe appears within the α-helical amino acid sequence or the Z₂ moiety. In alternative embodiments, the peptide contains not more than two consecutive aromatic amino acid residues positions along the length of the xx-helical amino acid sequence or along the length of the Z₂ moiety (or along the length of the peptide as a whole). For example, if multiple phenylalanine residues are present, -Phe-Phe- may be present within the α-helical amino acid sequence or the Z₂ moiety, but-Phe-Phe-Phe will not be present. In another embodiment, the peptide contains no more than three or, alternatively, no more than two aromatic amino acid residues along the length of the peptide; and preferably the peptide contains not more than two consecutive aromatic amino acid residues.

The N-terminal amino acid is covalently attached to Z₁, which is a moiety comprising an aromatic group or a fluorophore. The aromatic group can be an aryl or heteroaryl, and may include single, multiple, or fused ring structures.

In certain embodiments, where Z₁ comprises the aromatic group, the aromatic group is selected from the group consisting of phenylacetyl, naphthylacetyl, fluorenylacetyl, pyrenylacetyl, and cinnamoyl. Other aromatic groups can optionally be used to promote self-assembly.

In certain embodiments, where Z₁ comprises the fluorophore, the fluorophore is 4-nitro-2,1,3-benzoxadiazolyl (“NBD”), 5-(dimethylamino)naphthalene-1-sulfonyl, 4-(N,N-dimethylaminosulfonyl)-2,1,3-benzoxadiazolyl, or 9-acridinyl. Other fluorophore groups can optionally be used to promote fluorescence and, optionally, self-assembly.

In certain embodiments, it may also be desirable to include a single amino acid residue within the α-helical amino acid sequence, or within the Z₂ moiety, which allows for one or more agents to be conjugated to the peptide by coupling via side chains of amino acids, including the amino group of lysine, the guanidine group of arginine, the thiol group of cysteine, or the carboxylic acid group of glutamic acid or aspartic acid.

In general, amino groups present in lysine side chains can be reacted with reagents possessing amine-reactive functional groups using known reaction schemes. Exemplary amine-reactive functional groups include, without limitation, activated esters, isothiocyanates, and carboxylic acids. Examples of conjugating a chemotherapeutic agent (e.g., doxorubicin, daunorubicin, taxol) to a Lys sidechain are described in DeFeo-Jones et al., Nature Med. 6(11): 1248-52 (2000), Schreier et al., PlosOne 9(4):e94041 (2014), Gao et al., J Am Chem Soc. 131:13576 (2009), each of which is hereby incorporated by reference in its entirety.

In general, guanidine groups present in arginine can be reacted with reagents possessing guanidine-reactive groups using known reaction schemes. Exemplary guanidine-reactive functional groups include, without limitation, NHS esters using gas phase synthesis (McGee et al., J. Am. Chem. Soc., 134 (28):11412-11414 (2012), which is hereby incorporated by reference in its entirety).

In general, thiol groups present in cysteine (or cysteine derivative) side chains can be reacted with reagents possessing thiol-reactive functional groups using known reaction schemes. Exemplary thiol-reactive functional groups include, without limitation, iodoacetamides, maleimides, and alkyl halides.

In general, carboxyl groups present in glutamic or aspartic acid side chains, or at the C-terminal amino acid residue, can be reacted with reagents possessing carboxyl-reactive functional groups using known reaction schemes. Exemplary carboxyl-reactive functional groups include, without limitation, amino groups, amines, bifunctional amino linkers.

In each of the types of modifications described above, it should be appreciated that the conjugate can be directly linked via the functional groups of the peptide and the reagent to be conjugated, or via a bifunctional linker that reacts with both the peptide functional groups and the functional groups on the reagent to be conjugated.

In certain embodiments, the peptides of the invention include naphthyl-(CH₂)—C(O)— or NBD-(CH₂)₂—C(O)— (also referred to herein as NBD-βAla-) as the Z₁ moiety, and phosphotyrosine or tyrosine as the Z₂ moiety.

Exemplary peptides of the present invention include, without limitation:
naphthyl-(CH₂)—C(O)-Leu-Leu-Leu-Leu-(p)Tyr (see SEQ ID NO: 11 where Xaa at position 5 is phospho-Tyrosine);
NBD-(CH₂)₂—C(O)-Leu-Leu-Leu-Leu-(p)Tyr (see SEQ ID NO: 2 where Xaa at position 6 is phospho-Tyrosine);
naphthyl-(CH₂)—C(O)-Leu-Leu-Leu-Leu-Tyr (see SEQ ID NO: 11 where Xaa at position 5 is Tyrosine);
NBD-(CH₂)₂—C(O)-Leu-Leu-Leu-Leu-Tyr (see SEQ ID NO: 2 where Xaa at position 6 is Tyrosine);
naphthyl-(CH₂)—C(O)-Ile-Ile-Ile-Ile-(p)Tyr (see SEQ ID NO: 12 where Xaa at position 5 is phospho-Tyrosine);
NBD-(CH₂)₂—C(O)-Ile-Ile-Ile-Ile-(p)Tyr (see SEQ ID NO: 13 where Xaa at position 6 is phospho-Tyrosine);
naphthyl-(CH₂)—C(O)-Ile-Ile-Ile-Ile-Tyr (see SEQ ID NO: 12 where Xaa at position 5 is Tyrosine);
NBD-(CH₂)₂—C(O)-Ile-Ile-Ile-Ile-Tyr (see SEQ ID NO: 13 where Xaa at position 6 is Tyrosine);
naphthyl-(CH₂)—C(O)-Val-Val-Val-Val-(p)Tyr (see SEQ ID NO: 14 where Xaa at position 5 is phospho-Tyrosine);
NBD-(CH₂)₂—C(O)-Val-Val-Val-Val-(p)Tyr (see SEQ ID NO: 15 where Xaa at position 6 is phospho-Tyrosine);
naphthyl-(CH₂)—C(O)-Val-Val-Val-Val-Tyr (see SEQ ID NO: 14 where Xaa at position 5 is Tyrosine); and
NBD-(CH₂)₂—C(O)-Val-Val-Val-Val-Tyr (see SEQ ID NO: 15 where Xaa at position 6 is Tyrosine).
   To the extent that specific embodiments are not otherwise listed above, it is explicitly contemplated that each of the peptides listed above may contain L-amino acids.

A further aspect of the invention relates to a self-assembled product formed by exposing the peptide to a phosphatase that is suitable to cause dephosphorylation of the phosphorylated amino acid residue. In certain embodiments, the self-assembled product is in the form of an oligomerized product that includes two or more peptides of the invention in dephosphorylated form. The dephosphorylated peptides co-assemble during oligomerization and hydrogelation. Preferably, each of the two or more peptides have an alpha-helix structure.

In certain embodiments, the oligomerization and hydrogelation occurs in an aqueous environment, in which case the resulting product takes the form of a supramolecular hydrogel formed upon self-assembly of the activated peptide(s) of the invention in an aqueous medium. As described herein, the term “supramolecular hydrogel” refers to a network of nanofibers or nanoribbons formed by the self-assembly of peptides as the solid phase to encapsulate water (Du et al., Chem. Asian J. 9(6): 1446-1472 (2014), which is hereby incorporated by reference in its entirety).

A further aspect of the invention relates to a pharmaceutical composition comprising a pharmaceutically acceptable carrier and a peptide or oligomerized product of the invention.

According to one embodiment, two or more of the peptides are present.

In some embodiments, the carrier is an aqueous medium. In one embodiment, the aqueous medium is a sterile isotonic aqueous buffer, which is typically well tolerated for administration to an individual. Additional exemplary aqueous media include, without limitation, normal saline (about 0.9% NaCl), phosphate buffered saline (“PBS”), sterile water/distilled autoclaved water (“DAW”), as well as cell growth medium (e.g., MEM, with or without serum), aqueous solutions of dimethyl sulfoxide (“DMSO”), polyethylene glycol (“PEG”), and/or dextran (less than 6% per by weight.)

To improve patient tolerance to administration, the pharmaceutical composition may have a pH of about 5 to about 8. In one embodiment, the pharmaceutical composition has a pH of about 6.5 to about 7.4. In some embodiments, the sodium hydroxide or hydrochloric is added to the pharmaceutical composition to adjust the pH.

In other embodiments, the pharmaceutical composition includes a weak acid or salt as a buffering agent to maintain pH. Citric acid has the ability to chelate divalent cations and can thus also prevent oxidation, thereby serving two functions as both a buffering agent and an antioxidant stabilizing agent. Citric acid is typically used in the form of a sodium salt, typically 10-500 mM. Other weak acids or their salts can also be used.

The pharmaceutical composition may also include solubilizing agents, preservatives, stabilizers, emulsifiers, and the like. A local anesthetic (e.g., lidocaine, benzocaine, etc.) may also be included in the compositions, particularly for injectable forms, to ease pain at the site of the injection.

In some embodiments, the peptide or peptides may each be present at a concentration of about 1 μM to about 10 mM, about 10 μM to about 5 mM, about 50 M to about 2 mM, or about 100 μM to about 1 mM, such as from about 100 μM to about 500 μM. The volume of the composition administered, and thus, dosage of the peptide administered can be adjusted by one of skill in the art to achieve optimized results. In one embodiment, between 100 and about 800 μg can be administered per day, repeated daily or periodically (e.g., once every other day, once every third day, once weekly). This can be adjusted lower to identify the minimal effective dose, or tailored higher or lower according to the nature of the treatment being effected.

Additional aspects of the invention relate to administering one or more peptides or compositions or hydrogels of the invention to a subject to promote a desired effect. In these various embodiments, administering may be carried out topically, intraperitoneally, intralesionally, ocularly, intraocularly, intranasally, orally, rectally, transmucosally, intranasally, intradermally, intestinally, parenterally, intramuscularly, subcutaneously, intravenously, intraarterially, intramedullary by implantation, by intracavitary or intravesical instillation, intrathecally, as well as direct intraventricular, intraperitoneal, intrasynovially, by intraocular injection, or by introduction into one or more lymph nodes. Administration can be repeated periodically during the course of a treatment regimen, for example, one or more times per week, daily, or even one or more times per day.

In some embodiments, the subject is a mammal. Suitable mammals include, without limitation, rodents, rabbits, canines, felines, ruminants, and primates such as monkeys, apes, and humans. In one embodiment, the subject is a human.

The products and compositions of the present invention afford a number of uses.

In one aspect, the invention relates to a method of causing cell death that includes contacting a cell that overexpresses a phosphatase with one or more peptides of the invention, or a pharmaceutical composition containing the same, which one or more peptides is phosphorylated, whereby said contacting is effective to cause uptake of the one or more peptides and dephosphorylation of the phosphorylated amino acid residue thereof by the phosphatase and thereby allow for intracellular self-assembly of the dephosphorylated one or more peptides.

In certain embodiments, the cell that overexpresses the phosphatase is a cancer cell. The cancer cells to be treated in accordance with these aspects can be present in a solid tumor, present as a metastatic cell, or present in a heterogenous population of cells that includes both cancerous and noncancerous cells.

Exemplary cancer conditions include, without limitation, cancers or neoplastic disorders of the brain and CNS (glioma, malignant glioma, glioblastoma, astrocytoma, multiforme astrocytic gliomas, medulloblastoma, craniopharyngioma, ependymoma, pinealoma, hemangioblastoma, acoustic neuroma, oligodendroglioma, meningioma), pituitary gland, breast (Infiltrating, Pre-invasive, inflammatory cancers, Paget's Disease, Metastatic and Recurrent Breast Cancer), blood (Hodgkin's Disease, Leukemia, Multiple Myeloma, Lymphoma), lymph node cancer, lung (Adenocarcinoma, Oat Cell, Non-small Cell, Small Cell, Squamous Cell, Mesothelioma), skin (melanoma, basal cell, squamous cell, Kapsosi's Sarcoma), bone cancer (Ewing's Sarcoma, Osteosarcoma, Chondrosarcoma), head and neck (laryngeal, pharyngeal, and esophageal cancers), oral (jaw, salivary gland, throat, thyroid, tongue, and tonsil cancers), eye, gynecological (Cervical, Endrometrial, Fallopian, Ovarian, Uterine, Vaginal, and Vulvar), genitourinary (Adrenal, bladder, kidney, penile, prostate, testicular, and urinary cancers), and gastrointestinal (appendix, bile duct (extrahepatic bile duct), colon, gallbladder, gastric, intestinal, liver, pancreatic, rectal, and stomach cancers).

In this embodiment, the cancer cells to be treated can be either ex vivo or in vivo.

In certain embodiments, the cell that overexpresses the phosphatase is an induced pluripotent stem cell (iPSC). iPSCs, including human iPSCs, may serve as promising materials for regenerative therapy. However, as discussed above, their ability to undergo unlimited self-renewal and pluripotent differentiation makes iPSCs tumorigenic after transplantation. Therefore, complete differentiation or selective elimination of residual undifferentiated cells is essential for the clinical application of these derivatives.

The iPSC to be treated in accordance with these aspects can be present in a heterogenous population of cells that includes both the iPSCs and differentiated cells. This embodiment is particularly useful when carrying out an autologous transfer of cells that have been modified ex vivo, and then treated in accordance with the present invention prior to administration of such modified cells to the individual from whom they were initially obtained. In this manner, the contacting step is desirably carried out ex vivo.

A further aspect of the invention relates to a method for selectively causing cell death in a mixed population of cells. This method includes providing a mixed population of cells including differentiated cells and one or more iPSCs; and contacting the mixed population of cells with one or more peptides of the invention, or a pharmaceutical composition containing the same, which one or more peptides is phosphorylated, whereby said contacting is effective to cause uptake of the one or more peptides and dephosphorylation of the phosphorylated amino acid residue thereof by a phosphatase overexpressed by the induced pluripotent stem cells, and thereby allow for intracellular self-assembly of the dephosphorylated one or more peptides in the induced pluripotent stem cells, but not differentiated cells, and selective induction of cell death in the induced pluripotent stem cells containing intracellular self-assemblies of the dephosphorylated one or more peptides.

The ex vivo contacting step is preferably carried out for less than 2 hours, such as from about 30 minutes up to about 120 minutes, including about 30 to about 45 minutes, about 45 to about 60 minutes, about 60 to about 75 minutes, about 75 to about 90 minutes, about 90 to about 105 minutes, or about 105 minutes to about 120 minutes. Further, the contacting step is preferably carried out using a peptide present at a concentration defined above, preferably from about 100 μM to about 1 mM, such as from about 200 μM to about 800 μM. Persons of skill in the art will be able to optimize the peptide dose and duration of treatment depending on the level of ALPs expressed by the iPSCs. For example, where iPSCs express higher levels of ALPs, then a lower concentration of peptide may be used for shorter duration.

A further aspect relates to a population of differentiated cells recovered from the processes of the present invention, which population of differentiated cells is free of iPSCs and, thus, suitable for transplantation into a patient. Such a population of differentiated cells can be used for autologous transplant procedures or heterologous transplant procedures.

According to another aspect, the invention relates to a method of treating a patient for cancer or inhibiting cancer cell efflux of an antineoplastic agent, anticancer drug, or chemotherapeutic drug.

According to one embodiment, the method of treating cancer includes administering to the patient a peptide of the invention or a pharmaceutical composition containing the same. The administering the peptide allows cancer cells to take up the peptide, or an oligomerization product formed by the peptide, which selectively causes cell death of cancer cells that overexpress phosphatase enzymes. Numerous cancer types have been previously demonstrated to overexpress alkaline phosphatase, including those noted above.

While any class of antineoplastic agent, anticancer drug, or chemotherapeutic drug is contemplated for use in combination with the present invention, exemplary agents within these classes include alkylating agents, platinum drugs, antimetabolites, anthracycline and non-anthracycline antitumor antibiotics, topoisomerase inhibitors, and mitotic inhibitors, corticosteroids and targeted cancer therapies (such as imatinib, Gleevec®; gefitinib, Iressa®; sunitinib, Sutent®; and bortezomib, Velcade®).

According to one embodiment, the method of treating cancer includes administering to the patient an antineoplastic agent, an anticancer drug, or a chemotherapeutic drug; and administering to the patient a solution comprising a peptide of the invention. These steps of administering the agents/drugs and peptide allows cancer cells to take up the peptide, or an oligomerization product formed by the peptide, and the administered agents/drugs.

As a consequence of administering the agents/drugs and peptide, the peptide or oligomerization product inhibits efflux of the antineoplastic agent, anticancer drug, or chemotherapeutic drug from cancer cells, further enhancing cancer cell death as compared to the peptide alone.

### EXAMPLES

The examples below are intended to exemplify the practice of embodiments of the disclosure but are by no means intended to limit the scope thereof.

### Materials & Methods for Examples 1-8

Materials and Instruments: 2-Cl-trityl chloride resin (1.0-1.2 mmol/g), HOBt, HBTU, Fmoc-OSu, and other Fmoc-amino acids were purchased from GL Biochem (Shanghai, China). Other chemical reagents and solvents were purchased from Fisher Scientific. Alkaline phosphatase was purchased from Biomatik (Cat. No. A1130, Alkaline Phosphatase [ALP], 30000 U/mL, in 50% Glycerol.), Fetal bovine serum (FBS) and penicillin-streptomycin were purchased from Gibco by Life Technologies. All precursors were purified with Agilent 1100 Series Liquid Chromatograph system, equipped with an XTerra C18 RP column and Variable Wavelength Detector. The LC-MS spectra were obtained with a Waters Acquity Ultra Performance LC with Waters MICROMASS detector, and ¹HNMR spectra on Varian Unity Inova 400. Circular dichroism (CD) spectra were obtained with a Jasco J-810 Spectropolarimeter. UV-Vis spectra were obtained with a Varian Cary 50 Bio UV-Visible Spectrophotometer.

TEM Sample Preparation: After placing 5 μL samples on 400 mesh copper grids coated with continuous thick carbon film (˜35 nm) which is glowed discharged, we washed the grid with ddH₂O and UA (uranyl acetate). The sample loaded grid was stained with the UA. The residual UA was removed by filter paper and then dried in air. TEM images were obtained with Morgagni 268 transmission electron microscope.

Critical Micelle Concentration ((MC) Measurement: The CMCs were determined using pyrene as the fluorescent probe. Different concentrations of compounds were prepared in pyrene-saturate solutions. The fluorescence spectra of pyrene solutions with different concentration compounds were obtained. The intensity ratio of 378 nm/393 nm (I₃₇₈/I₃₉₃) was determined by a Synergy H1 hybrid multi-mode microplate reader. Plot I₃₇₈/I₃₉₃ against the concentrations of compounds. The concentration at the turning point is the CMC.

Dephosphorylation Rate Measurement: To 100 μL solution of 5, 7 or 9 in PBS, ALP was added, and the mixtures were shaken at 37° C. At different time point, 900 μL methanol was added to quench the enzyme reaction. The reaction mixtures were analyzed with LC-MS.

Cell Culture: Human induced pluripotent stem cell line A21 was generated from human normal dermal fibroblasts by using the StemRNA™-NM Reprogramming kit (Stemgent, Cat #00-0076). hiPSCs were routinely cultured and passaged on 6-well plates coated with 0.25 μg/cm² iMatrix-511 (Recombinant Laminin-511) (ReproCell) with NutriStem XF/FFTM medium (Biological Industries). HS-5 cell line and HEK293 cell line were purchased from American Type Culture Collection (ATCC, USA). HS-5 cells were cultured in Dulbecco's Modified Eagle Medium (DMEM) supplemented with 10% fetal bovine serum (FBS), 100 U/mL penicillin and 100 μg/mL streptomycin. HEK293 cells were cultured in Minimal Essential Medium (MEM) supplemented with 10% fetal bovine serum (FBS), 100 U/mL penicillin and 100 μg/mL streptomycin. All cells were maintained at 37° C. in a humidified atmosphere of 5% CO₂.

Differentiation of Human iPSC to iPS-derived Hematopoietic Progenitor Cells (HPC's): iPSCs were differentiated into hematopoietic progenitor cells (HPCs) by using a 3D-bioreactor platform¹. HPCs released from iPSC-spheroids after 9-10 days' differentiation were collected and characterized. Hematopoietic lineage specific marker expression of harvested HPCs were analyzed by flow cytometry. About 97.6% of these HPCs were CD31+CD43+ double positive, but only about 13% are OCT4+, indicative of commitment to hematopoietic lineage. These iPS-derived HPCs were used for 5 (400 μM, 2 hr) cytotoxicity assay.

Cell Viability of iPSC's: iPSCs were plated in 6-well plates and incubated for 24 to 48 hours, then media were replaced with fresh one (2 ml) supplemented with PBS (control) 5 (200 μM, 300 μM, and 400 μM), 7 (400 μM) or 9 (400 μM), and incubated for 2 hours. Media were removed and cells were rinsed with PBS once, fresh normal cultural media were added and incubated for 30 min. All cells were collected and stained with trypan blue, live cells were counted using Cellometer Auto 2000 (Nexcelom Bioscience). Data were obtained by from three independent wells (n=3).

Cell Viability of iPS-derived HPC's: iPS-derived HPCs were plated in 12-well plates and incubated overnight, then media were replaced with fresh one (2 ml) supplemented with PBS (control) 5 (400 μM) incubated for 2 hours. All cells were collected and stained with trypan blue, live cells were counted using Cellometer Auto 2000 (Nexcelom Bioscience). Data were obtained by from three independent wells (n=3).

Cell Viability of Differentiated Cells: Cytotoxicity against HS-5 cells and HEK293 cells was determined by using MTT assay. Cells were seeded in 96-well plates at 1×10⁵ cells/well for 24 hours followed by culture medium removal and subsequently addition of culture medium containing different concentration of 5 (immediately diluted from fresh prepared 10 mM stock solution). After 1/2/3 hours, the culture medium with 5 was replaced by fresh culture medium and 10 μL MTT solution (5 mg/mL) was added to each well and incubated at 37° C. for another 4 h. Then 100 μL of SDS-HCl solution was added to stop the reduction reaction and dissolve the formazan. The absorbance of each well at 595 nm was measured by a DTX880 Multimode Detector. The results were calculated as cell viability percentage relative to untreated cells. Data were obtained by from three independent wells (n=3).

Confocal Laser Scanning Microscopy (CLSM) Imaging: For live cell imaging, cells in exponential growth phase were seeded in a confocal dish (3.5 cm) at 1.0×10⁵ cells per dish and then incubated in incubator for 24 h. We removed culture medium, and added fresh medium containing precursors for different time points. After removing the medium and washing the cells with live cell imaging solution (2 mL×2), the cells were used for CLSM imaging. For time-dependent live cell imaging, cells in exponential growth phase were seeded in a confocal dish (3.5 cm) at 1.0×10⁵ cells per dish and then incubated in incubator for 24 h. After removing culture medium, we treated the cells with 1 mL of Hoechst 33342 (1 μg mL⁻¹) for 10 minutes. After being washed with culture medium (2 mL×2), the cells were incubated with fresh medium containing precursor 5 in a Tokai Hit stage top incubator (STXF-WSKMX-SET) to be used for CLSM imaging. All the CLSM images were obtained using Zeiss LSM 880 confocal microscopy at the lens of 63× with oil. The lasers used are 405 nm and 488 nm.

Degradation: 5 million HS-5 cells were made into 1 mL lysate by freeze-thaw lysis method. To 100 μL lysate, 5 (200 μM) and rhodamine 6G (inner standard) were added. The mixtures were shaken at 37° C. At different time point, 900 μL methanol was added to quench the reaction. The reaction mixtures were analyzed with LC-MS.

### Example 1-Synthesis and Characterization of the Precursors

Two precursors, NBD-β-Alanine and Fmoc-L-phospho-Tyrosine, were prepared for the subsequent synthesis of peptides 3, 5, 7, and 9. The synthesis of NBD-β-Alanine and Fmoc-L-phospho-Tyrosine are illustrated in Scheme 1 below.

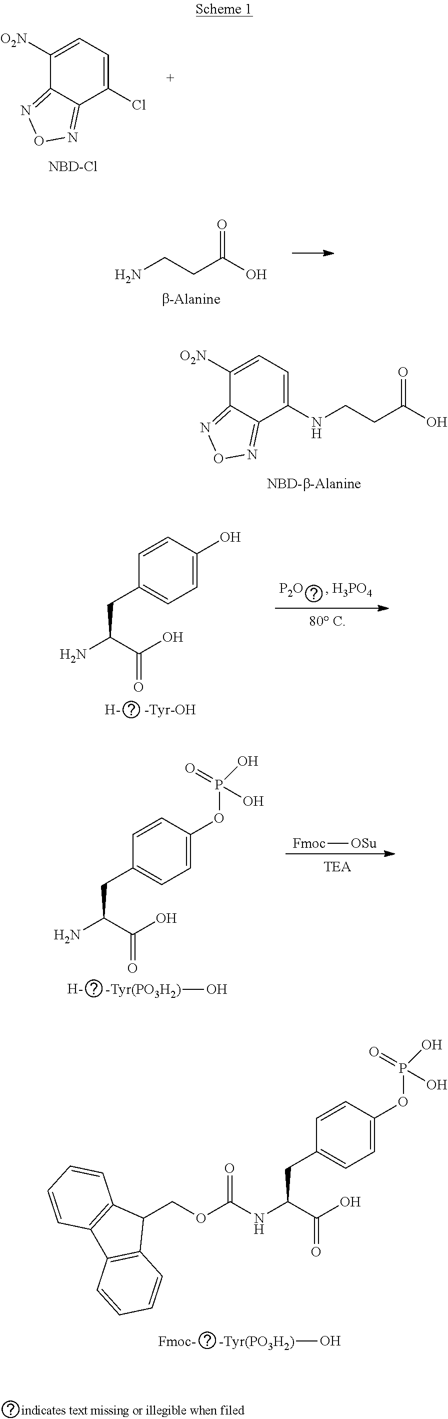

Synthesis of NBD-β-Alanine: To the solution of β-Alanine (5 mmol, 1 g) and K₂CO₃ (15 mmol, 2 g) in H₂O (15 mL), the solution of NBD-Cl in MeOH (30 mL) was added dropwise under the protection of N₂. After reaction at room temperature for 3 h, the MeOH was removed by evaporation. After adding 70 mL H₂O, the pH was the solution was adjusted by 1 M HCl to ˜3. The solution was extracted by diethyl ether (200 mL×3), and the organic part was dried by Na₂SO₄, filtered and concentrated by evaporation. The structure was confirmed by ¹H NMR and MS. ¹H NMR: (400 MHZ, CD₃OD-d₄) δ (ppm): 8.55 (m, 1H), 6.40 (d, 1H), 3.82 (s, 2H), 2.79 (t, 2H). MS: calc. [M-H]⁻=251.04, obsvd. ESI-MS: M/Z=250.95.

Synthesis of Fmoc-_{L}-Tyr(PO₃H₂)—OH: The mixture of P₂O₅ (35 mmol, 10 g), H₃PO₄ (133 mmol, 13 g) and H-_{L}-Tyr-OH (18 mmol, 3.22 g) was stirred for 24 h at 80° C. in N₂ atmosphere. After adding 30 mL H₂O and stirred for 30 min at 80° C., the reaction mixture was cool to room temperature. The reaction mixture was added to butanol (650 mL) dropwise and recrystallized at 4° C. overnight, filtration provided H-_{L}-Tyr(PO₃H₂)—OH as white power. To the solution of H-_{L}-Tyr(PO₃H₂)—OH (2 mmol, 522 mg) in H₂O (20 mL), the solution of Fmoc-OSu (2.4 mmol, 808 mg) in MeCN (20 mL) was added. After adjusting pH to ˜8 by triethylamine (TEA), the solution was stirred at room temperature for 2 h. After removal of MeCN by evaporation, 60 mL H₂O was added and the pH of the solution was adjusted to ˜3 by 1 M HCl. After extraction by ethyl acetate (100 mL×3), the organic part was washed by 1 M HCl (100 mL×2) and brine (100 mL×1). After being dried by Na₂SO₄, filtered and concentrated by evaporation, Fmoc-_{L}-Tyr(PO₃H₂)—OH was provided as white powder.

### Example 2-Synthesis and Characterization of Peptides 3, 5, 6, 7 and 9

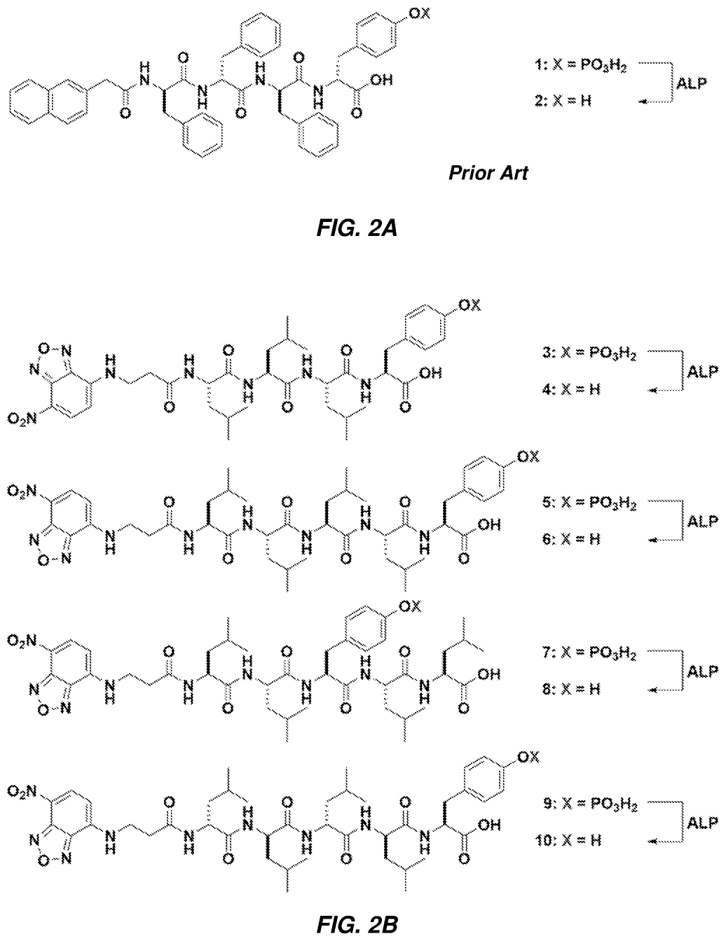

In the previously reported study (Kuang et al., “Efficient, Selective Removal of Human Pluripotent Stem Cells via Ecto-Alkaline Phosphatase-Mediated Aggregation of Synthetic Peptides,” Cell Chem. Biol. 24(6):685-694.e4 (2017), which is hereby incorporated by reference in its entirety), the substrate for EISA was a D-phosphotetrapeptide (1, FIG. 2A), consisting of a 2-naphthylacetyl group to enhance π-π interactions, a D-Phe-D-Phe-D-Phe as the β-sheet forming motif, and a D-phosphotyrosine as the trigger for ALP-catalyzed dephosphorylation. After enzymatic dephosphorylation, 1 becomes 2 (FIG. 2A). As reported by Kuang et al., the self-assembly of 2 forms β-sheet, which occurs pericellularly on iPSCs and selectively kills iPSCs.

To minimize the formation of β-sheets and to maintain hydrophobicity for self-assembly in water, L-leucine (Leu)—which is known to have high helix propensity (Lyu et al., “Alpha-helix Stabilization by Natural and Unnatural Amino Acids with Alkyl Side Chains,” Proc. Natl. Acad. Sci. U.S.A. 88(12):5317-5320 (1991), which is hereby incorporated by reference in its entirety)—was instead selected as the amino acid for constructing the peptide backbone. To visualize the location of the peptide assemblies in cellular environment after EISA, the 2-naphthylacetyl group was replaced with NBD (4-nitro-2,1,3-benzoxadiazole, an environment-sensitive fluorophore) that is particularly useful for revealing peptide assemblies in cells (Feng et al., “Artificial Intracellular Filaments,” Cell Rep Phys Sci 1(7): 100085 (2020); Gao et al., “Imaging Enzyme-triggered Self-assembly of Small Molecules Inside Live Cells,” Nat Commun 3:1033 (2012), each of which is hereby incorporated by reference in its entirety). It was not known a priori, however, whether the Leu-rich peptides would self-assemble and selectively kill iPSCs.

Based on the foregoing, the phosphopeptide, NBD-βA-LLLₚY (3) (see SEQ ID NO: 1), was designed and synthesized in the manner described above. Although 3 is able to turn into 4 upon dephosphorylation catalyzed by ALP (see FIG. 2B), 4 only exhibits limited self-assembling ability. Thus, the number of leucine in 3 was increased to make NBD-βA-LLLLₚY (SEQ ID NO: 2) (5, FIG. 2B). For further understanding the self-assembly of this leucine-rich sequence, two other phosphopentapeptides, 7 and 9, were also designed. In NBD-βA-LLₚYLL (7) (SEQ ID NO: 3), L-phosphotyrosine was moved from the C-terminal position to the middle. By placing tyrosine between two dileucine motifs, the de-phosphorylated peptide, 8, was expected to be less prone for α-helix conformation because tyrosine is less prone to form helix than leucine (Pace et al., “A Helix Propensity Scale Based on Experimental Studies of Peptides and Proteins,” Biophys. J. 75(1):422-427 (1998), which is hereby incorporated by reference in its entirety). In another analog, the C-terminal position of L-phosphotyrosine was maintained, but replaced L-Leu in 5 was replaced with D-Leu. Such a change results in NBD-βA-llllₚY (9, lowercase “L” represents D-Leu). Phosphopeptide 9 and its corresponding de-phosphorylated pentapeptide (10) are heterochiral peptides, which was expected to help understand the contribution of homochirality to the morphology of the peptide assemblies.

Based on the molecular design shown in FIG. 2B, solid phase peptide synthesis (SPPS) was used to make the phosphopeptides (3, 5, 7, and 9; Scheme 2). Briefly, after the reaction between NBD-Cl and β-alanine to produce NBD-β-alanine (Cai et al., “Environment-Sensitive Fluorescent Supramolecular Nanofibers for Imaging Applications,” Anal. Chem. 2014, 86 (4), 2193-2199 (2014), which is hereby incorporated by reference in its entirety) and protecting L-phosphotyrosine by 9-fluorenylmethoxycarbonyl (Fmoc) (see Example 1, supra), the designed precursors were synthesized via SPPS using Fmoc-protected amino acids (Fmoc-_{L}-Tyr(PO₃H₂)—OH, Fmoc-_{L}-Tyr(tBu)-OH, Fmoc-_{L}-Leu-OH, Fmoc-_{D}-Leu-OH), 2-Cl-trityl chloride resin, NBD-β-alanine, HOBt, and HBTU. Dephosphorylated peptide 6 was obtained following treatment of 5 with ALP. After purification by high performance liquid chromatography (HPLC), peptides 3, 5, 6, 7 and 9 were obtained as yellow power.

The structures of peptides 3, 5, 6, 7, and 9 were confirmed by ¹H NMR and LC-MS as follows:

MS of 3: calc. [M-H]⁻=833.32, obsvd. ESI-MS: M/Z=833.61.

¹H NMR of 5 (400 MHZ, DMSO-d₆) δ (ppm): 7.77 (d, 1H, J=12 Hz), 7.14 (d, 2H, J=8 Hz), 7.04 (d, 2H, J=8 Hz), 6.45 (d, 1H, J=8 Hz), 4.36 (m, 1H), 4.28 (m, 4H), 2.98 (m, 2H), 2.86 (m, 2H), 2.61 (m, 2H), 1.54 (m, 3H), 1.41 (m, 8H), 1.25 (m, 1H), 0.79 (m, 24H).

MS of 5: calc. [M-H]⁻=946.41, obsvd. ESI-MS: M/Z=946.63.

MS of 6: calc. [M-H]⁻==866.44, obsvd. ESI-MS: M/Z=866.52.

¹H NMR of 7 (400 MHZ, DMSO-d₆) δ (ppm): 7.72 (d, 1H, J=12 Hz), 7.08 (d, 2H, J=8 Hz), 7.02 (d, 2H, J=8 Hz), 6.44 (d, 1H, J=12 Hz), 4.36 (m, 1H), 4.28 (m, 4H), 2.98 (m, 2H), 2.80 (m, 2H), 2.62 (m, 2H), 1.52 (m, 2H), 1.39 (m, 8H), 1.24 (m, 2H), 0.79 (m, 24H). MS of 7: calc. [M-H]⁻=946.41, obsvd. ESI-MS: M/Z=946.60.

¹H NMR of 9 (400 MHZ, DMSO-d₆) δ (ppm): 7.75 (m, 1H), 7.12 (d, 2H, J=8 Hz), 7.00 (d, 2H, J=8 Hz), 6.44 (d, 1H, J=8 Hz), 4.46 (m, 1H), 4.23 (m, 4H), 2.98 (m, 2H), 2.75 (m, 2H), 2.61 (m, 2H), 1.48 (m, 12H), 0.81 (m, 24H).

MS of 9: calc. [M-H]⁻=946.41, obsvd. ESI-MS: M/Z=946.53.

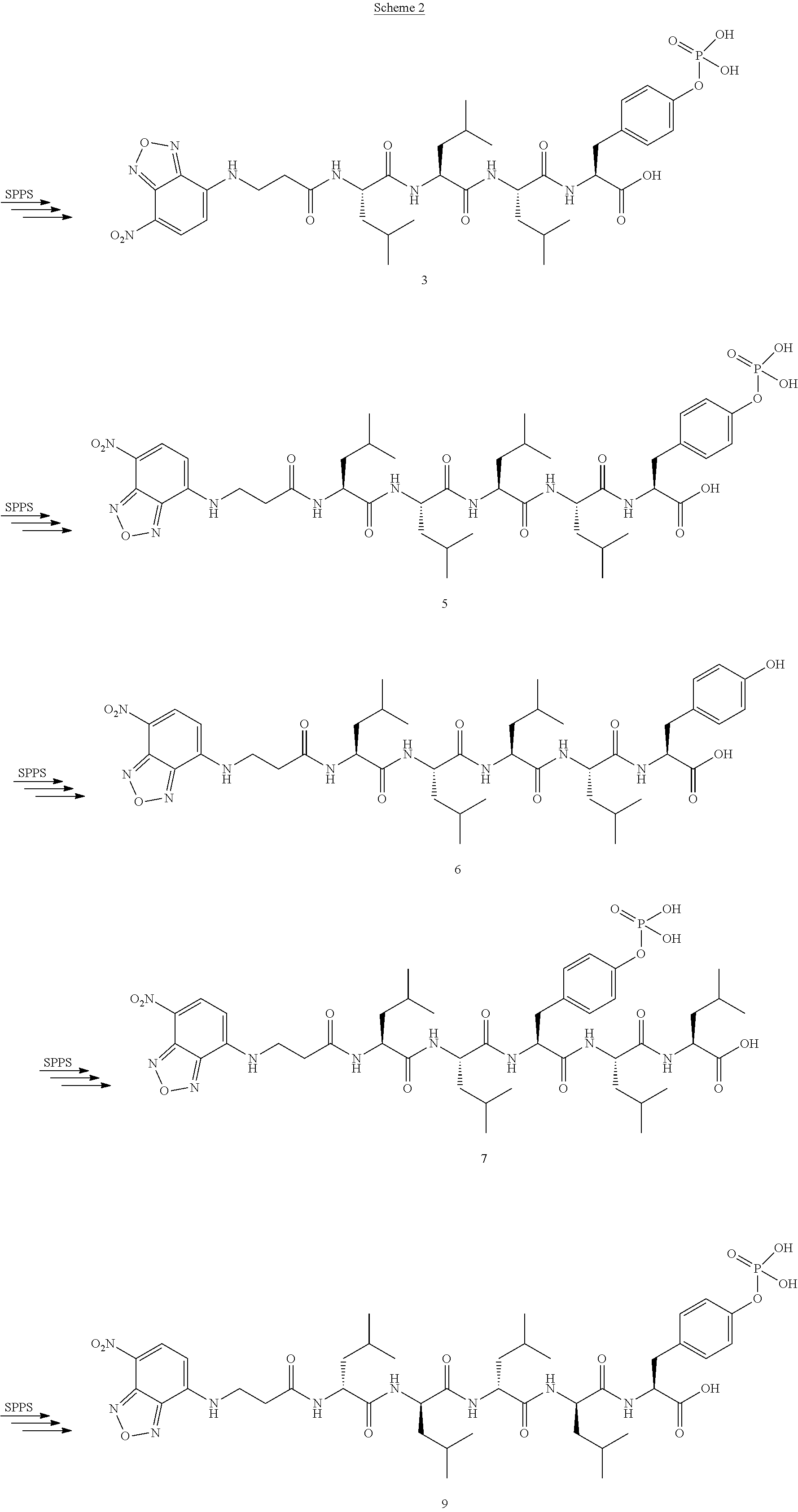

### Example 3-Enzymatic Self-Assembly In Vitro

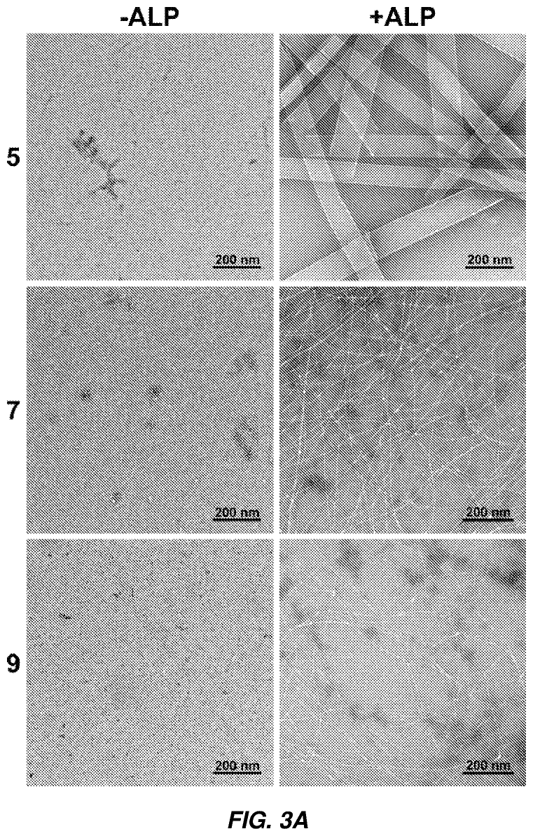

After obtaining all the precursors, their behaviors for EISA were evaluated in vitro using transmission electron microscopes (TEM) to examine the nanostructures formed before and after the ALP catalyzed dephosphorylation of precursors 5, 7, and 9. At 400 μM and in phosphate buffered saline (PBS), 5 self-assembles to form short nanofibers with the diameter of 9±2 nm and a few nanoparticles (FIG. 3A). The critical micelle concentration (CMC) of 5 is 86.1 μM. In the solution of 7 (400 μM) and 9 (400 μM), TEM images show nanoparticles only, and the CMCs of 7 and 9 are 159.9 UM and 146.8 μM, respectively. These results indicate that, among the three phosphopentapeptides, 5 exhibits higher self-assembling ability than 7 or 9 does. After the solution of 5 (400 μM) was incubated with ALP (0.5 U/mL) at 37° C. for 24 h, TEM shows that dephosphorylation of 5 results in nanoribbons with the widths of 74±13 nm. FIG. 3B is a TEM image of 5, which shows the folded nanoribbons, indicating the self-assembly nanostructures are more like nanoribbons than nanotubes. Moreover, the lack of features of nanotubes, such as circular cross profiles, double layer of tubes, and the broken ends, in the TEM images suggest that the morphology of the assemblies of 6 likely is curved nanoribbons. The CMC of 6 is 8.1 μM. Unlike the case of 5, dephosphorylation of 7 or 9 at 400 μM by ALP (0.5 U/mL) produces nanofibers with the diameters of 6±2 nm or 9±2 nm, whose CMC is 87.4 μM or 25.8 μM, respectively.

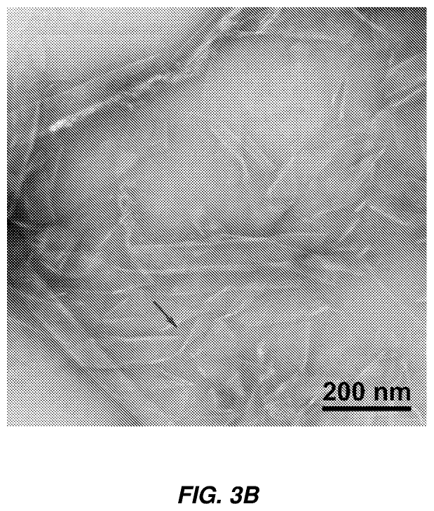

A key requirement for selectively eliminating iPSCs by EISA is that the L-peptide nanoribbons only form on and in iPSCs, which overexpress ALP, but not on and in the differentiated cells that express normal level of ALP. That is, L-peptide nanoribbons should only rapidly form at high level of ALP, but not at normal level of ALP. Considering that the normal level of ALP in serum is about 0.1 U/mL (Burtis et al., Tietz Textbook of Clinical Chemistry and Molecular Diagnostics, Elsevier Saunders (2006), which is hereby incorporated by reference in its entirety) and abnormally high level ALP can be 0.6-0.8 U/mL (Abdallah et al., “Serial Serum Alkaline Phosphatase as an Early Biomarker for Osteopenia of Prematurity,” Medicine (Baltimore) 95(37):e4837 (2016); Gibson et al., “Clinical Problem-solving; Out of the Blue,” N Engl J Med 370(18): 1742-8 (2014), each of which is hereby incorporated by reference in its entirety), EISA of 5 was tested in the presence of different concentrations ALP (from 0.1 U/mL to 0.8 U/mL) for 1 hour and 2 hour (FIG. 4). Peptide 5 was chosen for more in depth experimentation, because it selectively kills iPSCs (vide infra). One hour after the addition of ALP in the solution of 5 (400 μM), TEM shows that nanofibers form in the presence of 0.1 U/mL of ALP, the mixture of nanofibers and nanoribbons form at 0.2 and 0.4 U/mL of ALP, with more nanofibers at 0.2 U/mL of ALP and more nanoribbons at 0.4 U/mL of ALP. When the concentrations of ALP are at 0.6 and 0.8 U/mL, TEM shows dominantly nanoribbons. After two hours of incubation of the solution of 5 (400 μM) with ALP, TEM shows that dephosphorylation of 5 results in nanoribbons when the concentrations of ALP are 0.1, 0.2, 0.4, 0.6, and 0.8 U/mL. Notably, decreasing the concentration of 5 to 200 μM, one-hour treatment by 0.1 U/mL or 0.8 U/mL of ALP results in nanoparticles with the diameters of 404±50 nm or the nanoribbons, respectively. These results indicate that the initial concentration of 5, the expression levels of ALP, and the time of dephosphorylation control morphology of the nanoscale assemblies of the L-pentapeptides formed by EISA, similar to the case that the ratio of substrates and enzymes controls the rheological properties of hydrogels made by EISA (Guo et al., The Ratio of Hydrogelator to Precursor Controls the Enzymatic Hydrogelation of a Branched Peptide,” Soft Matter 2020, 16 (44), 10101-10105 (2020), which is hereby incorporated by reference in its entirety). Thus, after assessing the level of ALP expression in iPSCs, it should be possible to optimizing the initial concentration of a peptide and the time of dephosphorylation, to promote EISA that selectively targets iPSCs.

### Example 4-Enzymatic Conversion

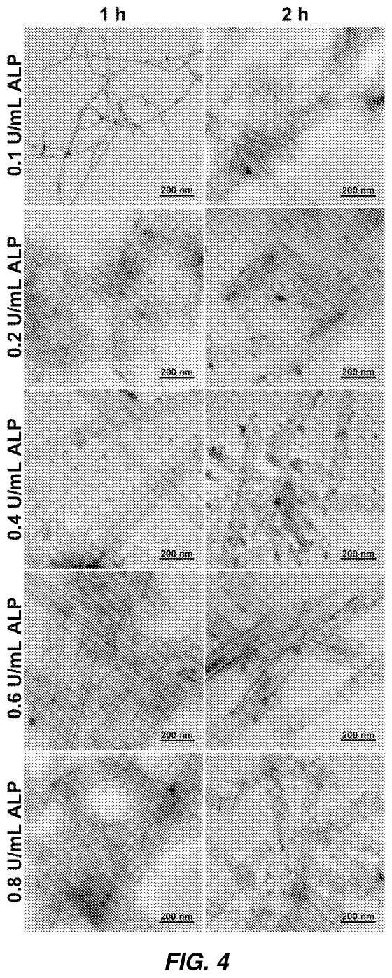

The morphological differences shown in FIGS. 3 and 4 imply that the extent of enzymatic reactions likely controls the self-assembly. Thus, the conversion of the phosphopentapeptides to their corresponding peptides was examined over time. Phosphopeptides 5, 7, or 9 were incubated at the concentration of 100, 200 or 400 μM with 0.5 U/mL ALP, and their dephosphorylation ratio was measured at different time points (FIG. 5). This concentration of ALP (0.5 U/mL) was selected, because iPSCs overexpress ALP (Stefkova et al., “Alkaline Phosphatase in Stem Cells,” Stem Cells Int 2015:628368 (2015), which is hereby incorporated by reference in its entirety) and nanoribbons of 5 formed within 1 hour when the ALP is >0.4 U/mL (FIG. 4). FIG. 5A shows the half-lives of 5 at 100, 200 and 400 μM are 27.3, 21.6. and 30.6 minutes, respectively. After 5 hours of incubation, 400 μM of 5 results in a dephosphorylation ratio of 93.8%, indicating that the nanoribbons, formed after 24 hours of incubation of 5 with ALP (FIG. 3, top row, right), mainly consist of 6. Decreasing the ALP concentration to 0.1 U/mL, the half-life of 5 (400 μM) is 122.2 min, and the final dephosphorylation ratio is 69.1% (FIG. 5B). After 1 hour of incubation, 32.7% of 5 turns into 6, and long nanofibers were observed (FIG. 4, first row, left). After 2 hours of incubation of 5 with 0.1 U/mL of ALP, 49.6% of 5 becomes 6, and TEM shows dominantly nanoribbons (FIG. 4, first row, right).

When incubated with 0.5 U/mL of ALP, 7 has a slower dephosphorylation rate than 5; the half-lives of 7 are 127.0, 112.0, and 125.5 minutes at 100, 200, and 400 μM, respectively (FIG. 5C). After 5 h of incubation, the conversion ratio of 7 to 8 is 61.5%, which results in nanofibers (FIG. 3, middle row, right). These results indicate that the phosphotyrosine in the middle of the phosphopentapeptide slows down the dephosphorylation catalyzed by ALP. Moreover, the assemblies of 7 likely hampers the interaction of phosphotyrosine with ALP. Having the D-leucine as the backbone and L-phosphotyrosine at the C-terminal of the pentapeptide, 9 exhibits a similar dephosphorylation rate as that of 5, with the half-lives of 24.6, 21.6 and 27.0 minutes at 100, 200, and 400 μM, respectively (FIG. 5D). After 5 hours of incubation, the conversion ratio of 9 to 10 is 93.3%, which results in nanofibers (FIG. 3, bottom right). These results and TEM in FIG. 3 indicate that the sequence of the pentapeptides also controls the morphology of nanostructures after the enzymatic reaction approaches completion.

### Example 5-Circular Dichroism Analysis

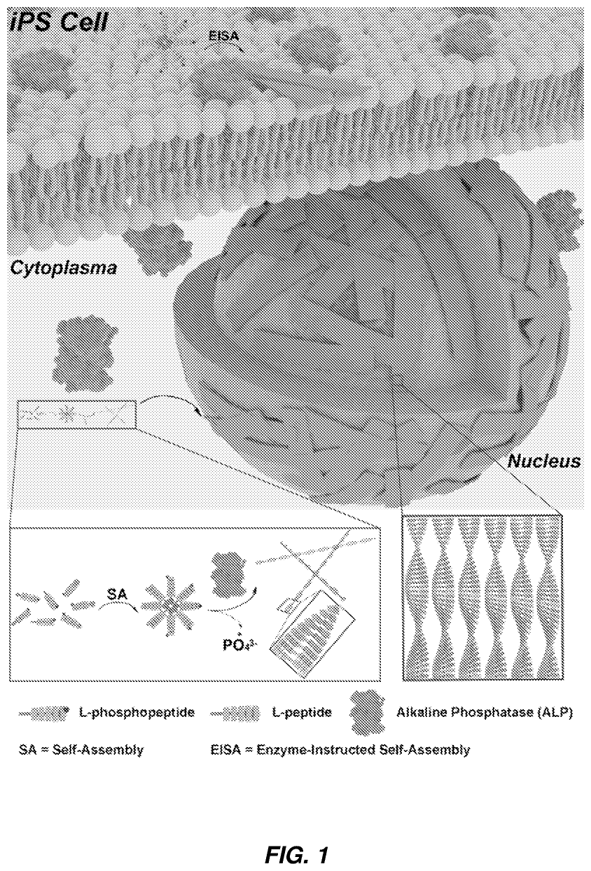

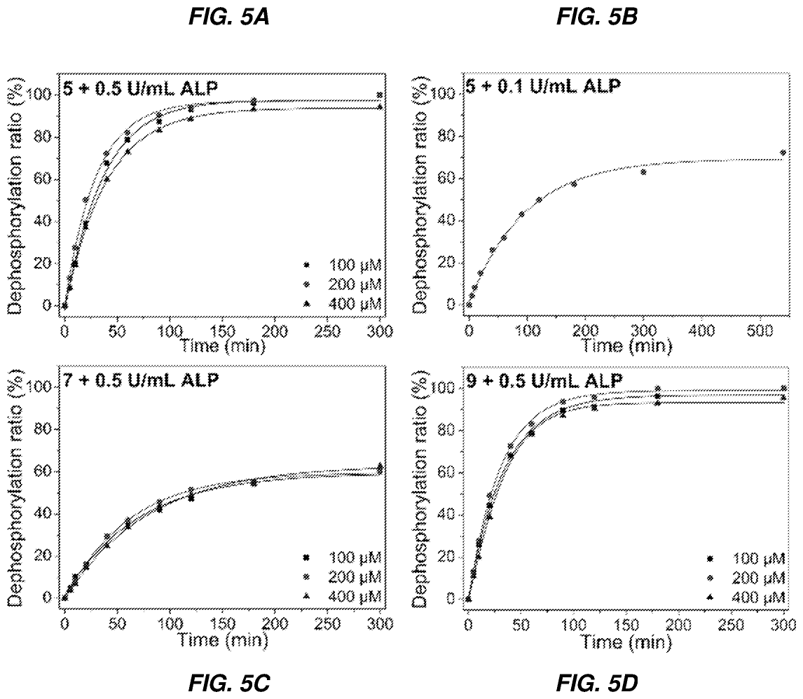

To understand the secondary structures of the L-phosphopentapeptide (5) in the assemblies without dephosphorylation and L-pentapeptide (6, NBD-βA-LLLLY, SEQ ID NO: 2) in the assemblies formed by the dephosphorylation of 5, the circular dichroism (CD) spectra of 5 was measured at different concentrations before and after the addition of ALP. The CD spectra of 5 at the concentrations ranging from 100 to 800 μM show slight negative trough at 225-250 nm (FIG. 6A, inset), which may be caused by discrete electronic transition (Rippon et al., “The 225-240-nm Circular Dichroism Band in Disordered and Charged Polypeptides,” Macromolecules 6(2):282-285 (1973), which is hereby incorporated by reference in its entirety) or a small amount of helix (Myer, “The pH-Induced Helix-Coil Transition of Poly-L-lysine and Poly-L-glutamic Acid and the 238-mu Dichroic Band,” Macromolecules 2(6):624-628 (1969), which is hereby incorporated by reference in its entirety). The CD band is weak because the phosphate group increases the solubility of 5, thus disfavoring the formation of extensive peptide assemblies that would enhance the CD signals. After the addition of ALP (0.5 U/mL) in the solution of 5, dephosphorylation of 5 produces 6, which exhibits two positive CD-bands at 204 and 229 nm, indicating the presence of α-helical conformation. The molar CD intensity of the two CD bands start to decrease when the concentration is higher than 400 μM, agreeing with the observation that 6, at high concentrations, aggregates to form precipitates. For further understanding the conformation of 6, we dissolve 6 in the mixture of trifluoroethanol (TFE) and PBS (TFE/PBS=1/1, pH 7.4), because TFE stabilizes secondary-structures (Roccatano et al., “Mechanism By Which 2,2,2-Trifluoroethanol/Water Mixtures Stabilize Secondary-structure Formation in Peptides: A Molecular Dynamics Study,” Proc. Nat'l Acad. Sci. USA 99(19): 12179-12184 (2002), which is hereby incorporated by reference in its entirety). The CD spectra show a positive peak at 199 nm and two negative peaks at 217 nm and 233 nm, which is the classical CD spectrum of α-helix. Since TFE favors the formation of α-helical conformation, further examination of the conformation of 5 and 6 was carried out by infrared spectroscopy. The Fourier-transform infrared (FTIR) spectra of 20 mM 5 and 6 show the peak near 1453 cm⁻¹ in the amide A/amide III region, which is caused by N—H stretch modes. In the amide I range, the peak at 1655 cm⁻¹ is consistent with the α-helical conformation, and the peaks around 1606 and 1619 cm⁻¹ likely originate from aggregated strands (Bagińska et al., “Conformational Studies of Alanine-rich Peptide Using CD and FTIR Spectroscopy,” J. Pept. Sci. 14(3):283-289 (2008), which is hereby incorporated by reference in its entirety). An additional peak at 1672 cm⁻¹ is mainly due to TFA counterions bound to the peptide (Castelletto et al., “Alpha Helical Surfactant-like Peptides Self-assemble into pH-dependent Nanostructures,” Soft Matter 17(11):3096-3104 (2021), which is hereby incorporated by reference in its entirety). Although a pentapeptide is too short to observe a persistent secondary structure, the dimer of 6 (FIG. 1) would be a decapeptide sequence to allow the observation of α-helix component. Peptide 5 (100 μM) was incubated with ALP (0.1 U/mL) and the CD spectra was measured every 10 min (FIG. 6B). The CD-bands at 204 nm and 229 nm appear after 40 minutes incubation (FIG. 6B, inset). At the time point 40 minutes, about 24% of 5 is hydrolyzed by ALP (FIG. 5B). This result indicates that morphological switch (from the micelle to nanoribbon) or significant self-assembly occurs when the ratio of [6] and [5] is about ⅓ ([5]=75 μM and [6]=25 μM). Notably, although NBD exhibits an absorption band in its UV-Vis spectra at about 344 nm, there is little induced circular dichroism (ICD) at that position. This result indicates that the dipoles of NBD moieties arrange in antiparallel direction in the nanoribbons formed by 6 (FIG. 1).

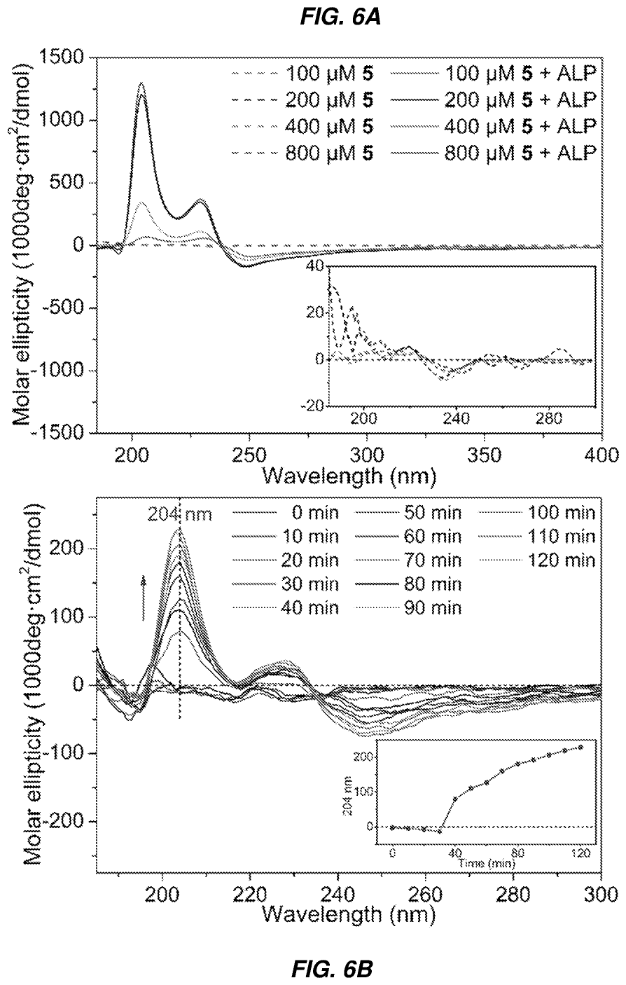

FIG. 7A shows the CD spectra of 7, the negative band at 202 nm indicates unordered structure (Gekko et al., “Vacuum-ultraviolet Circular Dichroism Analysis of Biomolecules,” Chirality 18(5):329-334 (2006), which is hereby incorporated by reference in its entirety). The dephosphorylated product (8, NBD-LLYLL) shows significant different CD troughs at different concentrations. When the concentration is 100 or 200 μM, a negative band around 220-250 nm was observed. When the concentration increases to 400 and 800 μM, the negative peak blue-shifts to 212 nm. Although the ICD-bands (300-350 nm) from NBD hardly show any constant trends, the presence of ICD band from NBD indicates that NBD moieties no longer orient in a manner to reduce dipole moments. Moreover, the CD signals of 8 are significantly lower than those of 6, indicating that the insertion of L-tyrosine between the two L-dileucines apparently weakens the ability of 8 to self-assemble. This observation agrees with TEM images of 8 showing thin nanofibers but not nanoribbons.

As shown in FIG. 7B, the CD spectra of 9 at the concentration ranging from 100 μM to 800 μM show two positive bands at 201 nm and around 223 nm, respectively, which likely originates from unordered structure (Gekko et al., “Vacuum-ultraviolet Circular Dichroism Analysis of Biomolecules,” Chirality 18(5):329-334 (2006), which is hereby incorporated by reference in its entirety). After the dephosphorylation of 9 to generate 10 (NBD-llllY), a negative band at 203 nm and positive bands around 220 nm and at 317 nm emerge. The negative band at 203 nm and positive band at 220 nm indicate the distorted α-helical conformation. The ICD bands from NBD show at 317 nm, indicating that NBD moieties arrange in a similar manner in the peptide assemblies of 10 formed at different concentrations. The CD signals of 10 also are weaker than those of 6, indicating that heterochirality disfavors self-assembly of 10. This observation also agrees with that 10 only forms thin nanofibers.

### Example 6-Selectively Killing iPSCs

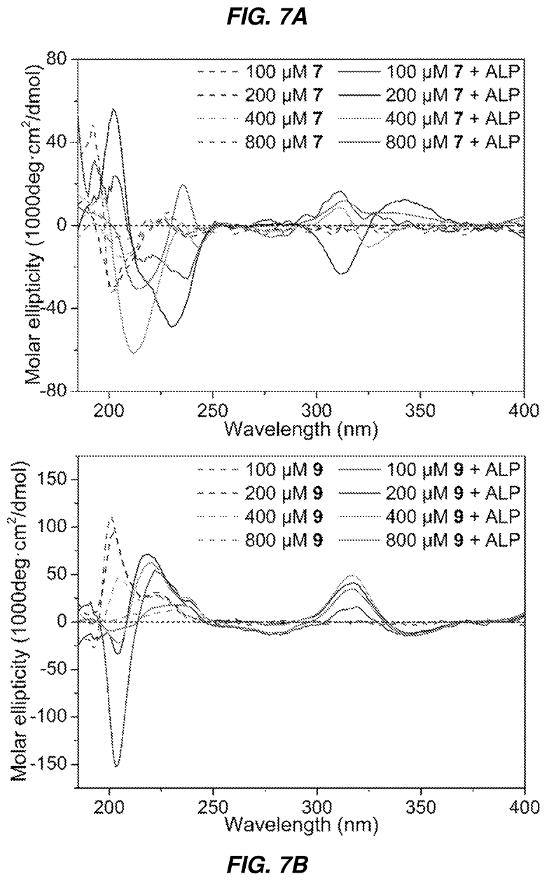

Peptides 5, 7 or 9 were incubated with iPSCs and cells counts were obtained using trypan blue staining. Incubation of 5 at the concentrations of 200, 300, and 400 μM with iPSCs (Takahashi et al., “Induction of Pluripotent Stem Cells from Mouse Embryonic and Adult Fibroblast Cultures by Defined Factors,” Cell 126(4):663-676 (2006), which is hereby incorporated by reference in its entirety) for 2 hours results in the cell viabilities of 31.05±3.20%, 18.50±1.50%, and 6.96±1.71%, respectively, confirming that 5 potently kills iPSCs (FIG. 8A). Such an activity is sufficient to prevent teratoma formation by iPSCs (Kuang et al., “Efficient, Selective Removal of Human Pluripotent Stem Cells via Ecto-Alkaline Phosphatase-Mediated Aggregation of Synthetic Peptides,” Cell Chem. Biol. 24(6):685-694.e4 (2017), which is hereby incorporated by reference in its entirety). Unlike 5, the other two phosphopentapeptides, 7 and 9, hardly exhibit cytotoxicity against the iPSCs at 400 μM (FIG. 8A). The difference of the cytotoxicity coincides with the significant different morphologies of the assemblies of 6, 8, and 10 formed by enzymatic dephosphorylation, but correlates less with the rates of enzymatic dephosphorylation of the phosphopeptides. This observation indicates that rapid formation of the nanoribbons (in this case, formed from 6) is ideal for killing iPSCs. To evaluate the cell selectivity of 5, the cytotoxicity of 5 was tested against normal cells by incubating 5 with HS-5 (bone marrow stromal cell) and HEK293 (embryonic kidney cell) (Roecklein et al., “Functionally Distinct Human Marrow Stromal Cell Lines Immortalized By Transduction with the Human Papilloma Virus E6/E7 Genes,” Blood 85(4): 997-1005 (1995); Su et al., “The Effect of Forced Growth of Cells into 3D Spheres Using Low Attachment Surfaces on the Acquisition of Stemness Properties,” Biomaterials 34(13):3215-3222 (2013), each of which is hereby incorporated by reference in its entirety). As shown in FIG. 8B, 5, at 400 μM, exhibits slight cytotoxicity towards HS-5 cells, likely due to the high expression of acid phosphatase inside HS-5 (Roecklein et al., “Functionally Distinct Human Marrow Stromal Cell Lines Immortalized By Transduction with the Human Papilloma Virus E6/E7 Genes,” Blood 85(4):997-1005 (1995), which is hereby incorporated by reference in its entirety). At 400 μM, 5 is innocuous to HEK293 cells (FIG. 8B). The low-level expression of ALP in HS-5 or HEK293 cells indicates that the selectivity of 5 towards iPSCs mainly originates from the ALP expression levels.

The cytotoxicity of 5 was tested against iPS-derived HPCs. After 9-10 days' differentiation, the HPCs were released from iPSC-spheroids and collected, and then the hematopoietic lineage specific marker expression of harvested HPCs was analyzed by flow cytometry. About 97.6% of these HPCs were CD31+CD43+ double positive, indicating that HPCs has high purity. After incubation with 5 (400 μM) for 2 h, the viability of the HPC cells is 96.8% (FIG. 8B). Moreover, significant morphology change (e.g., nuclei becoming much darker) of iPSCs occurs after treatment by 5 (400 μM, 2 h), but there is no significant morphology change on iPS-derived HPCs after the same treatment. In addition, considering the inherent heterogeneity of the iPSCs or the inherent heterogeneity of the HPCs obtained from differentiation, the efficacy of 5 in FIG. 8, in fact, confirms the efficiency and selectivity of 5 for eliminating iPSCs. That is, FIG. 8A shows 93% cell death, agreeing with that the iPSCs generated from A21 is about 94% iPSCs in total cell population. On the other hand, FIG. 8B shows 97% cell survival, agreeing with that the HPCs derived from iPSCs contains about 98% HPCs and 2% iPSCs in the total cell population. Thus, the percentages of cell killing (FIG. 7A) and cell survival (FIG. 8B) are consistent with the purity of iPSCs and HPCs in the total cell population. These results indicate that 5, by rapidly forming nanoribbons upon the dephosphorylation catalyzed by ALP, efficiently and selectively eliminates iPSCs in the mixed cell population made of iPSCs and non-iPSCs.

### Example 7-Evaluating Formation of Intranuclear Assemblies

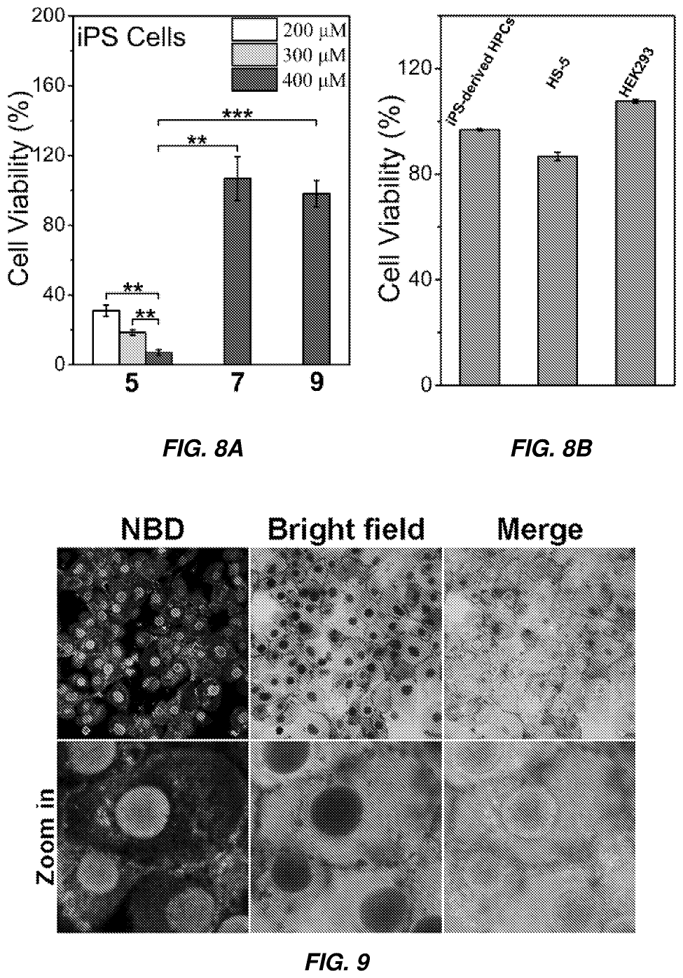

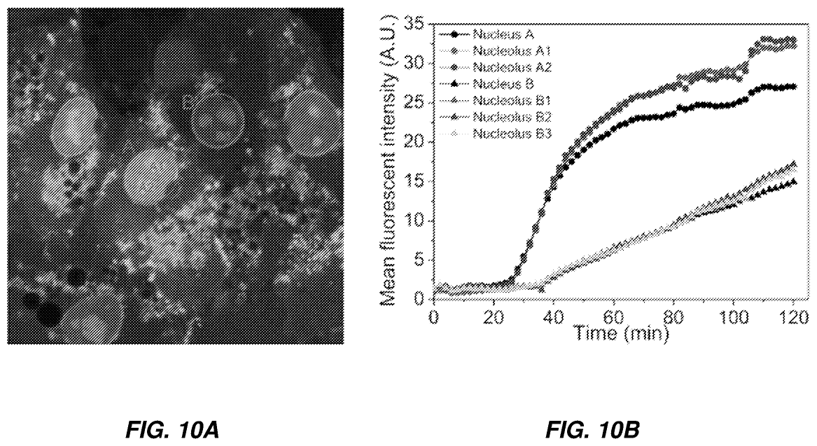

Because the fluorescence of NBD increases drastically from unassembled to assembled state, confocal laser scanning microscopy (CLSM) was suitable to reveal the cellular location of the peptide assemblies formed after dephosphorylation catalyzed by ALP. After incubation with 5 at 400 μM for 2 hours, the iPSCs exhibit strong NBD fluorescence in nuclei, and much weaker fluorescence in cytoplasm and on membrane except a few puncta (FIG. 9). Moreover, the bright field image shows black nuclei, as observed by optical microscope (FIG. 9). The blackness in nuclei overlaps well with the strong fluorescence. In addition, even stronger fluorescence and blackness is exhibited in nucleoli (FIG. 10), indicating that the EISA of 5 targets and aggregates in iPSC nuclei, especially in nucleoli. To understand how dephosphorylation affects nucleus-targeting, iPSCs were incubated with 5 (400 μM) and an inhibitor (2,5-Dimethoxy-N-(quinolin-3-yl)benzenesulfonamide (DQB), 5 μM) of tissue nonspecific alkaline phosphatase for 2 hours and the cells were examined by CLSM, which shows little fluorescence in cells. The results confirms that the nucleus-targeting mainly originates from the ALP catalyzed dephosphorylation. The nucleus-targeting is also related to the concentration of 5, because decreasing the concentration to 200 μM results in hardly any fluorescence in the cells. This result indicates that self-assembly to form micelles/nanoparticles of 5 is a predicate for nuclear targeting. Decreasing the treatment time to 1 hour leads to weaker fluorescence and light blackness in the nuclei in fluorescent and optical modes, respectively. Nevertheless, the nuclei still exhibit strong fluorescence, confirming nuclear accumulation of 6. iPSCs were also incubated with 7 or 9 at 400 μM for 2 hours. The incubation by 7 for 2 hours leads to no fluorescence inside the cells. As to the iPSCs treated by 9 for 2 hours, most of the cells exhibit no fluorescence inside the cells, several of them show NBD-fluorescence on membrane, and a few of them exhibit fluorescent puncta in nuclei, which indicates a slight inhomogeneity of the iPSCs populations in terms of the expression level of ALP.

To trace the dynamics of the formation and distribution of 5 inside iPSC cells, time-lapse CLSM was also used to image the changes of the fluorescence in the iPSCs incubated by 5 (400 μM). Using one cell as a representative case, after 6 minutes incubation fluorescent puncta appear on the membrane, likely originating from the aggregates of assemblies of 5 and those of 6 generated by the dephosphorylation catalyzed by ALP. This observation indicates that, as a surfactant-like peptide (Vauthey et al., “Molecular Self-assembly of Surfactant-like Peptides to Form Nanotubes and Nanovesicles,” Proc. Natl. Acad. Sci. USA 99(8):5355-5360 (2002), which is hereby incorporated by reference in its entirety), 5 firstly adheres to the cell membrane, and then is hydrolyzed by ALP to form 6. After 24 minutes incubation, fluorescence starts to grow in the cytoplasm. Considering the LLLLY motif in SEQ ID NO: 2 constitutes the transmembrane domains of 18 human membrane proteins, it is possible that the affinity of 5 or 6 to membrane allows the assemblies of the mixture of 5 and 6 to interact with cellular membranes, which then facilitates nuclear localization. At about 28 minutes of incubation, fluorescence appears in the nucleus. Moreover, the nuclei shrink after the assemblies emerge in the nuclei. In addition, the nuclei also show nuclear blebbing (Stephens et al., “Chromatin Histone Modifications and Rigidity Affect Nuclear Morphology Independent of Lamins,” Mol. Biol. Cell 29(2):220-233 (2018), which is hereby incorporated by reference in its entirety). The shrinkage of nuclei and nuclear blebbing likely associate with iPSC death. To further examine the dynamics of EISA-formed peptide assemblies in iPSCs over different time, the increase of fluorescence in two cells over 2 hours was monitored; their nuclei, A and B, are shown in FIG. 10A. FIG. 10B shows the mean fluorescence intensity in the two nuclei and their nucleoli. The mean fluorescent intensity in Nucleus A starts to increase at 28 minutes and levels off after 110 minutes. For Nucleus B, the mean fluorescent intensity keeps increasing from 38 minutes. The increases of the fluorescence in the nuclei and nucleoli are synchronous. These results indicate that the assemblies made of 5 and 6 firstly self-assemble on the membrane after ALP-dephosphorylation to form fluorescent puncta, and then the assemblies enter the cells and quickly enter nuclei. The assemblies inside the nuclei or near nuclear membrane induce nuclear blebbing and shrinkage of nuclei, which likely contributes to the cell death.

### Example 8-Degradation of the L-pentapeptides

As a L-peptide, 5 should be proteolytic susceptible to proteases, especially after it is converted to 6. Thus, the stability of 5 was tested in the lysate of HS-5. Cell lysate was prepared from five million HS-5 cells and resuspended in 1 mL, then incubated with 200 μM of 5. All of 5 disappears (transform to 6) and only about 20% of 6 remains after 2 h of incubation. After 4 hours incubation, only 6.01% of 6 remains. This result agrees with the results demonstrating that 5 hardly inhibits HS-5 cells, and confirms that:
(i) 5 or 6, as an L-peptide, is biodegradable; and
(ii) because it can be proteolytically hydrolyzed, 5, after killing iPSCs, likely would lead to less side effects than D-peptides (e.g., 2) to differentiated cells in the cell mixtures.

### Discussion of Example 1-8

The preceding Examples demonstrate that a L-leucine-rich phosphopentapeptide (5) rapidly and selectively kills iPSCs by generation of intranuclear peptide assemblies via ALP catalyzed enzymatic self-assembly. Because the morphology of the peptide assemblies is controlled by the level of ALP and concentration of the precursors, EISA of 5 is able to control cell fates according to both the levels of enzyme expression and precursor concentrations. Unlike molecules that localize in nuclei by positive charge (Cai et al., “Supramolecular “Trojan Horse” for Nuclear Delivery of Dual Anticancer Drugs,” J. Am. Chem. Soc. 139(8): 2876-2879 (2017), which is hereby incorporated by reference in its entirety), the L-leucine-rich phosphopeptide bears negative charges. Although the exact pathway for 5 enters the nuclei of iPSCs remains to be elucidated, it is believed that the assemblies of 5 likely cluster ALP on cell surface to facilitate cellular uptake. Then, further dephosphorylation by ALP leads to their endosomal escape before entering the nuclei of iPSCs. Such an unconventional mode of cellular uptake of phosphopeptide assemblies is recently demonstrated by overexpressing ALP on HEK293 cells (He et al., “Dynamic Continuum of Nanoscale Peptide Assemblies Facilitates Endocytosis and Endosomal Escape,” Nano Lett 21(9):4078-4085 (2021), which is hereby incorporated by reference in its entirety). Moreover, the shrinkage of nuclei and nuclear blebbing suggest that the rapid formation of nuclear assemblies of 6 may generate local oncotic pressure to contribute to the iPSC death. In addition, it is believed that the nuclear accumulation of 6, without involving canonical nuclear location sequences (Dingwall et al., “Nuclear Targeting Sequences—A Consensus?” Trends in Biochemical Sciences 16:478-481 (1991), which is hereby incorporated by reference in its entirety), implies a possible new mechanism for nucleocytoplamic transport. The results from the peptides 7 and 9 indicate that both the rate of the enzymatic reaction and the molecule structures (e.g., sequence and stereochemistry) control the morphology of the resulted peptide assemblies. Although the effect of 5 on function of normal cells and iPSC-derived cells remains to be determined, the rapid degradation of 5 or 6 as unassembled L-peptide by HS-5 cells indicate that the long-term effects of the 5 or 6 likely would be minimal. While EISA has frequently resulted in the peptide assemblies made of β-sheets, the exploration of α-helical peptides for EISA has received less attention. This work illustrates the potential of enzymatic noncovalent synthesis for generating peptide assemblies of α-helices, because considerable amount of studies has already established a useful pool of peptides for generating helical assemblies of peptides (Castelletto et al., “Alpha Helical Surfactant-like Peptides Self-assemble into pH-dependent Nanostructures,” Soft Matter 17(11):3096-3104 (2021); Rhys et al., “Navigating the Structural Landscape of De Novo α-Helical Bundles,” J. Am. Chem. Soc. 141(22):8787-8797 (2019); Wang et al., “Structural Analysis of Cross α-helical Nanotubes Provides Insight into the Designability of Filamentous Peptide Nanomaterials,” Nat. Commun. 12(1):407 (2021), each of which is hereby incorporated by reference in its entirety) and there is rich information of the transmembrane domains of proteins.

### SEQUENCE LISTING

Submitted with this application is a Sequence Listing in the form of an ASCII text (.txt) file, which is hereby incorporated by reference into the specification of the application. The ASCII text file (6 KB) was created on Jun. 22, 2022 and has the file name 147376_000691.txt.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. All of the features described herein (including any accompanying claims, abstract and drawings), and/or all of the steps of any method or process so disclosed, may be combined with any of the above aspects in any combination, except combinations where at least some of such features and/or steps are mutually exclusive. Additionally, the recited order of processing elements or sequences, or the use of numbers, letters, or other designations therefore, is not intended to limit the claimed processes to any order except as may be specified in the claims. These alterations, improvements, and modifications are intended to be suggested hereby, and are within the spirit and scope of the invention. Accordingly, the invention is limited only by the following claims and equivalents thereto.

1. An isolated peptide comprising the structure below: 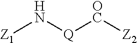
where
—NH-Q-C(O)— is an α-helical amino acid sequence comprising at least 4 and up to 30 amino acid residues,
Z₁ is a moiety comprising an aromatic group or a fluorophore,
Z₂ comprises a phosphorylated amino acid residue, or the dephosphorylated amino acid residue.

2. The isolated peptide according to claim 1, wherein the isolated peptide is 5 to 6 amino acids in length.

3. The isolated peptide according to claim 1, wherein the isolated peptide is 5 to 10 amino acids in length.

4. The isolated peptide according to claim 1, wherein the α-helical amino acid sequence comprises one or more residues independently selected from the group consisting of alanine, alpha-aminobutyric acid, norvaline, norleucine, and leucine.

5. The isolated peptide according to claim 4, wherein Q is:
(i) a tetrapeptide comprising -AA₁-AA₂-AA₃-AA₄- wherein each of AA₁, AA₂, AA₃, and AA₄ is independently selected from the group of alanine, alpha-aminobutyric acid, norvaline, norleucine, and leucine; or
(ii) a pentapeptide comprising -AA₁-AA₂-AA₃-AA₄-AA₅- wherein each of AA₁, AA₂, AA₃, AA₄ and AA₅ is independently selected from the group of alanine, alpha-aminobutyric acid, norvaline, norleucine, and leucine; or
(iii) a hexapeptide comprising -AA₁-AA₂-AA₃-AA₄-AA₅-AA₆- wherein each of AA₁, AA₂, AA₃, AA₄, AA₅, and AA₆ is independently selected from the group of alanine, alpha-aminobutyric acid, norvaline, norleucine, and leucine; or
(iv) a heptapeptide comprising -AA₁-AA₂-AA₃-AA₄-AA₅-AA₆-AA₇- wherein each of AA₁, AA₂, AA₃, AA₄, AA₅, AA₆, and AA₇ is independently selected from the group of alanine, alpha-aminobutyric acid, norvaline, norleucine, and leucine; or
(v) an octapeptide comprising -AA₁-AA₂-AA₃-AA₄-AA₅-AA₆-AA₇-AA₈- wherein each of AA₁, AA₂, AA₃, AA₄, AA₅, AA₆, AA₇, and AA₈ is independently selected from the group of alanine, alpha-aminobutyric acid, norvaline, norleucine, and leucine; or
(vi) a nonapeptide comprising -AA₁-AA₂-AA₃-AA₄-AA₅-AA₆-AA₇-AA₈-AA₉- wherein each of AA₁, AA₂, AA₃, AA₄, AA₅, AA₆, AA₇, AA₅, and AA₉ is independently selected from the group of alanine, alpha-aminobutyric acid, norvaline, norleucine, and leucine; or
(vii) a decapeptide comprising -AA₁-AA₂-AA₃-AA₄-AA₅-AA₆-AA₇-AA₈-AA₉-AA₁₀- wherein each of AA₁, AA₂, AA₃, AA₄, AA₅, AA₆, AA₇, AA&, AA₉, and AA₁₀ is independently selected from the group of alanine, alpha-aminobutyric acid, norvaline, norleucine, and leucine.

6-11. (canceled)

12. The isolated peptide according to claim 1, wherein Z₁ comprises the aromatic group.

13. The isolated peptide according to claim 12, wherein the aromatic group is selected from the group consisting of phenylacetyl, naphthylacetyl, fluorenylacetyl, pyrenylacetyl, and cinnamoyl.

14. The isolated peptide according to claim 1, wherein Z₁ comprises the fluorophore.

15. The isolated peptide according to claim 14, wherein the fluorophore is 4-nitro-2,1,3-benzoxadiazolyl (“NBD”), 5-(dimethylamino)naphthalene-1-sulfonyl, 4-(N,N-dimethylamino-sulfonyl)-2,1,3-benzoxadiazolyl, or 9-acridinyl.

16. The isolated peptide according to claim 1, wherein the phosphorylated amino acid residue at Z₂ is selected from the group of phospho-tyrosine, phospho-serine, and phospho-threonine.

17. The isolated peptide according to claim 1, wherein the peptide comprises L-amino acids.

18. The isolated peptide according to claim 5, wherein Z₁ is naphthyl-(CH₂)—C(O)— or NBD-(CH₂)₂—C(O)— and Z₂ is phosphotyrosine or tyrosine.

19. The isolated peptide according to claim 18, wherein the peptide is selected from the group consisting of:
(SEQ ID NO: 11)
naphthyl-(CH₂)-C(O)-Leu-Leu-Leu-Leu-(p)Tyr,
(SEQ ID NO: 2)
NBD-(CH₂)₂-C(O)-Leu-Leu-Leu-Leu-(p)Tyr,
(SEQ ID NO: 11)
naphthyl-(CH₂)-C(O)-Leu-Leu-Leu-Leu-Tyr,
(SEQ ID NO: 2)
NBD-(CH₂)₂-C(O)-Leu-Leu-Leu-Leu-Tyr,
(SEQ ID NO: 12)
naphthyl-(CH₂)-C(O)-Ile-Ile-Ile-Ile-(p)Tyr,
(SEQ ID NO: 13)
NBD-(CH₂)₂-C(O)-Ile-Ile-Ile-Ile-(p)Tyr,
(SEQ ID NO: 12)
naphthyl-(CH₂)-C(O)-Ile-Ile-Ile-Ile-Tyr,
(SEQ ID NO: 13)
NBD-(CH₂)₂-C(O)-Ile-Ile-Ile-Ile-Tyr,
(SEQ ID NO: 14)
naphthyl-(CH₂)-C(O)-Val-Val-Val-Val-(p)Tyr,
(SEQ ID NO: 15)
NBD-(CH₂)₂-C(O)-Val-Val-Val-Val-(p)Tyr,
(SEQ ID NO: 14)
naphthyl-(CH₂)-C(O)-Val-Val-Val-Val-Tyr,
and
(SEQ ID NO: 15)
NBD-(CH₂)₂-C(O)-Val-Val-Val-Val-Tyr

20. A supramolecular assembly of peptides according to claim 1, where at least a portion of the peptides are dephosphorylated.

21. (canceled)

22. A composition comprising one or more peptides according to claim 1 in an aqueous medium.

23. The composition according to claim 22, wherein peptide forms micelle structures/nanoparticles while the one or more peptides remain phosphorylated.

24. (canceled)

25. A method of causing cell death comprising:
contacting a cell that overexpresses a phosphatase with one or more peptides according to claim 1, or a composition comprising said one or more peptides, which one or more peptides is phosphorylated, whereby said contacting is effective to cause uptake of the one or more peptides and dephosphorylation of the phosphorylated amino acid residue thereof by the phosphatase and thereby allow for intracellular self-assembly of the dephosphorylated one or more peptides.

26. The method according to claim 25, wherein the cell that overexpresses the phosphatase is a cancer cell or an induced pluripotent stem cell.

27. (canceled)

28. The method according to claim 25, wherein the phosphatase is an alkaline phosphatase.

29-31. (canceled)

32. A method for selectively causing cell death in a mixed population of cells, comprising:
providing a mixed population of cells including differentiated cells and one or more induced pluripotent stem cells;
contacting the mixed population of cells with one or more peptides according to claim 1 or a composition comprising said one or more peptides, which one or more peptides is phosphorylated, whereby said contacting is effective to cause uptake of the one or more peptides and dephosphorylation of the phosphorylated amino acid residue thereof by a phosphatase overexpressed by the induced pluripotent stem cells, and thereby allow for intracellular self-assembly of the dephosphorylated one or more peptides in the induced pluripotent stem cells, but not differentiated cells, and selective induction of cell death in the induced pluripotent stem cells containing intracellular self-assemblies of the dephosphorylated one or more peptides.

33-35. (canceled)

36. The method according to claim 32 further comprising:
recovering a population of cells that is essentially free of, or free of, induced pluripotent stem cells.


SEQUENCE LISTING (15 sequences)

SEQ ID NO: 1 - protein, 5 aa, Artificial Sequence
  Peptide
  MISC_FEATURE (1)..(1): Xaa at position 1 is N-(4-nitro-2,1,3-benzoxadiazolyl)beta-Alanine
  MISC_FEATURE (5)..(5): Xaa at position 5 is phospho-Tyrosine or Tyrosine
XLLLX

SEQ ID NO: 2 - protein, 6 aa, Artificial Sequence
  Peptide
  MISC_FEATURE (1)..(1): Xaa at position 1 is N-(4-nitro-2,1,3-benzoxadiazolyl)beta-Alanine
  MISC_FEATURE (6)..(6): Xaa at position 6 is phospho-Tyrosine or Tyrosine
XLLLLX

SEQ ID NO: 3 - protein, 6 aa, Artificial Sequence
  Peptide
  MISC_FEATURE (1)..(1): Xaa at position 1 is N-(4-nitro-2,1,3-benzoxadiazolyl)beta-Alanine
  MISC_FEATURE (4)..(4): Xaa at position 4 is phospho-Tyrosine or Tyrosine
XLLXLL

SEQ ID NO: 4 - protein, 4 aa, Artificial Sequence
  Peptide
  1MISC_FEATURE (1)..(4): Xaa at positions (1)-(4) are each selected from alanine, alpha-aminobutyric acid, norvaline, valine, norleucine, isoleucine, and leucine
XXXX

SEQ ID NO: 5 - protein, 5 aa, Artificial Sequence
  Peptide
  METAL (1)..(5): Xaa at positions (1)-(5) are each selected from alanine, alpha-aminobutyric acid, norvaline, valine, norleucine, isoleucine, and leucine
XXXXX

SEQ ID NO: 6 - protein, 6 aa, Artificial Sequence
  Peptide
  MISC_FEATURE (1)..(6): Xaa at positions (1)-(6) are each selected from alanine, alpha-aminobutyric acid, norvaline, valine, norleucine, isoleucine, and leucine
XXXXXX

SEQ ID NO: 7 - protein, 7 aa, Artificial Sequence
  Peptide
  MISC_FEATURE (1)..(7): Xaa at positions (1)-(7) are each selected from alanine, alpha-aminobutyric acid, norvaline, valine, norleucine, isoleucine, and leucine
XXXXXXX

SEQ ID NO: 8 - protein, 8 aa, Artificial Sequence
  Peptide
  MISC_FEATURE (1)..(8): Xaa at positions (1)-(8) are each selected from alanine, alpha-aminobutyric acid, norvaline, valine, norleucine, isoleucine, and leucine
XXXXXXXX

SEQ ID NO: 9 - protein, 9 aa, Artificial Sequence
  Peptide
  MISC_FEATURE (1)..(9): Xaa at positions (1)-(9) are each selected from alanine, alpha-aminobutyric acid, norvaline, valine, norleucine, isoleucine, and leucine
XXXXXXXXX

SEQ ID NO: 10 - protein, 10 aa, Artificial Sequence
  Peptide
  MISC_FEATURE (1)..(10): Xaa at positions (1)-(10) are each selected from alanine, alpha-aminobutyric acid, norvaline, valine, norleucine, isoleucine, and leucine
XXXXXXXXXX

SEQ ID NO: 11 - protein, 5 aa, Artificial Sequence
  Peptide
  MISC_FEATURE (1)..(1): Xaa at position 1 is N-(naphthylacetyl)-Leucine
  MISC_FEATURE (5)..(5): Xaa at position 5 is phospho-Tyrosine or Tyrosine
XLLLX

SEQ ID NO: 12 - protein, 5 aa, Artificial Sequence
  Peptide
  MISC_FEATURE (1)..(1): Xaa at position 1 is N-(naphthylacetyl)- Isoleucine
  MISC_FEATURE (5)..(5): Xaa at position 5 is phospho-Tyrosine or Tyrosine
XIIIX

SEQ ID NO: 13 - protein, 6 aa, Artificial Sequence
  Peptide
  MISC_FEATURE (1)..(1): Xaa at position 1 is N-(4-nitro-2,1,3-benzoxadiazolyl)beta-Alanine
  MISC_FEATURE (6)..(6): Xaa at position 6 is phospho-Tyrosine or Tyrosine
XIIIIX

SEQ ID NO: 14 - protein, 5 aa, Artificial Sequence
  Peptide
  MISC_FEATURE (1)..(1): Xaa at position 1 is N-(naphthylacetyl)-Valine
  MISC_FEATURE (5)..(5): Xaa at position 5 is phospho-Tyrosine or Tyrosine
XVVVX

SEQ ID NO: 15 - protein, 6 aa, Artificial Sequence
  Peptide
  MISC_FEATURE (1)..(1): Xaa at position 1 is N-(4-nitro-2,1,3-benzoxadiazolyl)beta-Alanine
  MISC_FEATURE (6)..(6): Xaa at position 6 is phospho-Tyrosine or Tyrosine
XVVVVX